(19) United States
(12) Patent Application Publication
(10) Pub. No.: US 2021/0270831 A1
(43) Pub. Date: Sep. 2, 2021

(54) MODULAR BIOHYBRID SYSTEMS AND METHODS OF USE THEREOF

(71) Applicant: President and Fellows of Harvard College, Cambridge,, MA (US)

(72) Inventors: Junling Guo, Roxbury Crossing, MA (US); Miguel Suastegui, Boston, MA (US); Neel Saitish Joshi, Somerville, MA (US)

(21) Appl. No.: 17/255,757

(22) PCT Filed: Jun. 25, 2019
(86) PCT No.: PCT/US2019/038913

§ 371 Date: Dec. 23, 2020

Related U.S. Application Data
(60) Provisional application No. 62/691,397, filed on Jun. 28, 2018.

(51) Int. Cl. G01N 33/554; G01N 33/543

(52) U.S. Cl. CPC G01N 33/554; G01N 33/54346

(57) ABSTRACT

Modular biohybrid systems, some of which suitable for photochemical biosynthesis, are described. These systems are characterized by functionalized photocatalytic nanoparticles that are independently prepared, then assembled and attached to the modified surface of a cell, thereby enabling the cell to absorb light energy and convert it into chemical energy, for example in the form of a redox cofactor. The generated chemical energy then serves as fuel for production pathways of metabolites useful for the manufacturing of fuels, nutraceuticals, pharmaceuticals and cosmetics.

### CROSS REFERENCE TO RELATED APPLICATION

This application claims the benefit of priority from U.S. Provisional Patent Application No. 62/691,397, filed Jun. 28, 2018, the entire contents of which are incorporated herein by reference.

### STATEMENT OF GOVERNMENT SUPPORT

This invention was made with the U.S. government support under Grant No. DK110770 awarded by the National Institutes of Health. The government has certain rights in the invention.

### FIELD OF THE INVENTION

This disclosure relates to generally to biohybrid systems and methods thereof. More specifically, this disclosure relates to hybrid systems that incorporate biological systems (e.g., cells, tissue, whole organisms) with other organic or inorganic nanomaterials. Some of these systems are capable of harnessing and converting light energy into chemical energy to fuel biosynthesis of useful compounds.

### BACKGROUND

The rise of inorganic-biological hybrid systems is of widespread interest because of their distinct properties for the conversion of solar energy into chemical bonds, providing a sustainable and efficient biochemical synthesis platform. Significant strides toward comprehensive solar-to-chemical production have been demonstrated through several bioinorganic hybrid systems, including semiconductor conjugated with hydrogenases for biohydrogen production, long wavelength-absorbing nanomaterials integrated into plants for enhanced photosynthetic efficiency, and photoelectrodes coupled with whole cells for hydrogenation reactions and atmospheric CO₂ and N₂ fixation.

Microorganisms are already widely used in biomanufacturing because of their rapid proliferation and ability to convert renewable carbon sources into higher value chemicals through genetically programmable multi-step catalysis. In the context of inorganic-biological hybrids, autotrophic bacteria have been investigated intensively, and the breadth of metabolites produced using such approaches has been focused on relatively simple organic molecules. Although inorganic-biological hybrid systems based on autotrophic bacteria provide a sustainable, efficient, and versatile chemical synthesis platform. The available genetic engineering tools for autotrophic hosts are typically limited compared with the advanced toolboxes that exist for model heterotrophs. In other words, interfacing heterotrophs with inorganics may have its own advantages, especially in increasing efficiency for metabolic engineering efforts.

Sakimoto et al. (Science, 2016, 351(6268):74-77) describe the induced self-photosensitization of a native non-photosynthetic and CO₂-reducing bacterium, Moorella thermoacetica, with cadmium sulfide (CdS) nanoparticles, enabling the production of acetic acid from carbon dioxide. The CdS nanoparticles are generated in situ using exogenously added Cd²⁺ and cysteine as the sulfur source are precipitated by M. thermoacetica. However, not only is this system restricted to use of a specific bacterium including its metabolic pathways, the system's restriction to specific photocatalysts that the bacterium is capable of producing results in use of CdS nanoparticles that are cytotoxic.

Accordingly, a need still exists for biohybrid systems, including for biohybrid systems for photochemical biosynthesis.

### SUMMARY OF THE INVENTION

The present disclosure relates to biohybrid cell systems that are generally characterized by a biological cell having a chemically modified surface membrane and a plurality of functionalized nanoparticles that are prepared ex situ and then assembled on the modified cell surface membrane. In such hybrid systems that are capable of photochemical biosynthesis, photocatalytic nanoparticles are used on the cell surface to enable a biological cell to absorb and convert light energy into chemical energy, such as but not limited to binary semiconductor photocatalytic nanoparticles with a specific direct band gap range. Some embodiments of the hybrid system involve genetic modifications, for example genetic modifications to enhance one or more metabolic pathways to increase production of one or more desired metabolites, or genetic modifications to minimize loss of energy in the form of carbon atoms or metabolites having the same, adenosine triphosphate (ATP), redox cofactors, and electrons. In other embodiments, the nanoparticles assembled on the cell surface have fluorescent, radioactive, electromagnetic and/or magnetic properties, etc. The biohybrid cell systems of the invention are useful for the production of metabolites for the manufacturing of fuels, nutraceuticals, pharmaceuticals and cosmetics.

### BRIEF DESCRIPTION OF THE DRAWINGS

FIG. 1 is a rationally designed metabolic engineering scheme for overproduction of shikimic acid in the engineered strain S. cerevisiae Δzwf1. The parent strain S. cerevisiae Δzwf1 facilitates higher carbon flux in the non-oxidative pentose phosphate pathway (TKL1), which channels carbon toward shikimate. It also reduces carbon loss in the form of CO₂, but leads to a smaller pool of cytosolic NADPH.

FIG. 2 is a schematic diagram showing the assembly of S. cerevisiae-InP biohybrids, where the InP nanoparticles are first functionalized with polyphenol moieties, then assembled on the surface of genetically engineered yeast to form modular inorganic-biological hybrids.

FIG. 3 shows the band structure of InP and NADPH generation thermodynamic potential. The conduction band position of InP could drive the photocatalytic reduction of NADP⁺ to NADPH (E°=−0.324 V vs. NHE @ pH 7).

FIG. 4 shows the genetic cassettes that serves genetic knockout target candidates for overexpression of the key metabolic genes of shikimic acid biosynthesis in S. cerevisiae.

FIG. 5 is a schematic diagram depicting the modularization, assembly and interparticle locking of building blocks as described in Guo et al. (Nature Nanotechnology, 2016, 11:1105-1111), which has been adapted in the present invention but with modifications. This diagram depicts (i) polyphenol-based functionalization of versatile particles; and (ii) modular assembly of the polyphenol-functionalized particles to form core-satellite supraparticles with a core substrate. Modular assembly of building blocks on the core substrate is facilitated by interfacial molecular interactions between polyphenol moieties and the substrate, as well as interparticle “locking” via metal ligand coordination of polyphenol moieties.

FIG. 6 is a schematic diagram depicting biohybrid assembly process of the present disclosure.

FIG. 7A is a photograph of the setup of the photochemical production experiments. FIG. 7B is an infrared image of the photochemical production experimental setup of FIG. 7A. The temperatures were monitored by using advanced infrared camera FLIR E75 through the entire photochemical production experiments. FIG. 7C is a plot of temperature at different time points during an experiment. The temperature remains stable throughout the experiments. All points and error bars show the mean and error-propagated SD, respectively, of triplicate experiments.

FIGS. 8A-8C depict the characterization of the indium phosphide (InP) nanopowders used in the present invention. FIG. 8A shows the UV-Vis spectrum of InP nanopowder suspension in MQ water. The inset shows the photograph of the dried powders after the grinding process. FIG. 8B is an SEM image shows the morphology of InP nanopowders after grinding and separation process. FIG. 8C shows a collection of TEM images show the representative morphology of InP nanopowders with different shapes. Scale bars are 1 cm, 200 nm, and 20 nm for FIGS. 7A, 7B, and 7C, respectively.

FIGS. 9A-9F show the microstructure of S. cerevisiae Δzwf1-InP hybrid. FIG. 9A is a schematic model of S. cerevisiae Δzwf1-InP hybrid, consisting of InP nanoparticles assembled on the cell surface. FIGS. 9B and 9C are photographs of centrifuged samples of bare cells and S. cerevisiae Δzwf1-InP hybrids, respectively, in eppendorf tubes. The color change of pellet is ascribed to the assembly of InP nanoparticles on cells. FIG. 9C is shows the S. cerevisiae Δzwf1-InP hybrid being observed by TEM imaging of ultrathin cross-sectional specimen. The TEM image shows the overall picture of S. cerevisiae Δzwf1-InP hybrid and assembled InP shell with darker contrast. FIGS. 9E and 9F are TEM images of S. cerevisiae Δzwf1-InP hybrid magnified of FIG. 9D, where InP nanoparticles and cell microstructures can be observed. The small triangles highlight the position of individual InP nanoparticles. Scale bars are 500 nm, 100 nm, and 500 nm for FIGS. 9D, 9E, and 9F, respectively.

FIGS. 10A and 10B show the basic parameters of light source. FIG. 10A shows the spectrum of cold-white illumination source from circular LED array. FIG. 10B shows the intensity distribution of the LED in the plane located 100 mm from the LED along the emission axis. The intensity of the LED arrays is 3.0 mW/cm² as measured from 100 mm away along the emission axis. The data is obtained from Thorlabs (U.S.A.).

FIG. 11A shows a representative HPLC profile of 3-dehydroshikimic acid (DHS) observed at around 32 minutes with a 235 nm wavelength detection. FIG. 11B shows the representative HPLC profile of shikimic acid observed at around 25 minutes with a 210 nm wavelength detection. FIG. 11C shows a representative HPLC chromatogram of photochemical products by illuminated S. cerevisiae Δzwf1-InP hybrid suspensions after 72 hours fermentation. FIG. 11D shows a selected and magnified section of the HPLC chromatogram of FIG. 11C.

FIGS. 12A-12D present data from the physiological and metabolic characterization of the S. cerevisiae-InP hybrid system. FIG. 12A compares shikimic acid to dehydroshikimic acid (DHS) ratios in hybrids and in yeast only fermentations with light and dark conditions. FIG. 12B shows the total accumulation of shikimic acid (SA) and DHS after 72 h of growth. FIG. 12C shows the estimation of cytosolic-free NADPH/NADP⁺ ratio based on the conversion of SA to dehydroshikimic acid. FIG. 12D shows cell viability of various S. cerevisiae-InP hybrids and S. cerevisiae only strains based on counting of colony forming units (CFU). Insert shows the preparation of the bioinorganic hybrids does not affect initial CFU amount. The variation is represented by the standard deviation of three independent replicates in all graphs, *** (p-value <0.05). The engineered S. cerevisiae-InP bioinorganic hybrids were cultured for 72 hours in synthetic complete media lacking histidine and supplemented with 20 g L⁻¹ of glucose.

FIG. 13A shows a representative HPLC spectrum of photochemical products by S. cerevisiae Δzwf1 cells only suspensions in darkness after 72 hours fermentation. FIG. 13B is a selected and magnified section of the HPLC spectrum of FIG. 13A.

FIG. 14 shows shikimic acid to DHS production ratios of S. cerevisiae Δzwf1-InP hybrid compared to bare S. cerevisiae Δzwf1 cells. The variation is represented by the standard deviation of three independent replicates in all graphs; *** (p-value <0.05).

FIGS. 15A-15F present data associated with carbon utilization, cytosolic-free NADPH, and electron transfer in S. cerevisiae-InP hybrid system. FIG. 15A shows the glucose consumption of the course of a 72 hour broth culture. FIG. 15B shows the shikimic acid production profiles in light and dark conditions. Shikimic acid/DHS ratio, expressed as mass fraction. FIG. 15C shows the Specific shikimic acid yield based on consumed glucose and cell dry weight (CDW). FIG. 15D shows the percentage of variation in shikimic acid and byproduct formation of the bioinorganic hybrids under light (CL) versus dark (CD) conditions over time. FIG. 15E shows the proposed metabolic flux distributions based on total shikimic acid plus DHS concentrations and byproduct formation (glycerol and ethanol).

FIG. 15F shows the Differential pulse voltammetry of culture medium before and after growth experiment. Arrows indicate electrochemical signatures from possible suitable redox mediators capable of shuttling electrons to convert NADP⁺ to NADPH. Variation is represented as a standard deviation of three independent replicates in all graphs, *** (p-value <0.05).

FIG. 16A shows the kinetics of byproduct production from illuminated S. cerevisiae Δzwf1-InP hybrids. FIG. 16B shows the kinetics of byproducts production from S. cerevisiae Δzwf1-InP hybrids in darkness. Ethanol and glycerol were monitored by a HPLC equipped with a refractive index detector.

FIG. 17 shows the electrochemical characterization of cell medium. Differential pulse voltammetry based on glass carbon electrode of the growth medium before (fresh) and after (spent) fermentation. Arrows indicate peaks from possible redox mediators with sufficient reduction potential to drive NADP⁺ to NADPH conversion.

FIG. 18A is a schematic diagram depicting the experimental protocol for determining the possible origins of electron transfer mediators.

FIG. 18B shows shikimic acid to DHS production ratios of S. cerevisiae Δzwf1-InP hybrids subjected to the experimental protocol depicted in FIG. 18A.

FIGS. 19A-19E show the modular assembly of S. cerevisiae-polystyrene (PS) biohybrids. FIG. 19A is a schematic demonstration of engineering of S. cerevisiae-PS biohybrids, where the PS particles are fluorescent. FIG. 19B is a fluorescence microscopy image revealing the coreshell structure of S. cerevisiae-PS biohybrids. FIGS. 19C and 19D are reconstructed 3D images of S. cerevisiae-PS biohybrids from fluorescent confocal microscopy. FIG. 19E is TEM image of the cell surface of S. cerevisiae-PS biohybrids. Scale bars are 10 μm for FIG. 19B, 4 μm for FIG. 19C, 1 μm for FIG. 19D, and 500 nm for FIG. 19E.

FIGS. 20A-20E shows the modular assembly of S. cerevisiae-TiO₂ biohybrids. FIG. 20A is a schematic demonstration of assembly of S. cerevisiae-TiO₂ biohybrids. FIG. 20B is an SEM image showing the TiO₂ nanoparticles assembled on the yeast cell surface and the cell retained the native shape. FIGS. 19C-19E are EDS mapping images show the high-angle annular dark-field scanning transmission electron microscopy (HAADF-SEM), Ti, and Fe element mapping images of S. cerevisiae-TiO₂ biohybrids. The Ti signal corresponds to the presence of TiO₂ nanoparticles, and the Fe signal is ascribed to the use of Fe in the polyphenol-based particle functionalization and interparticle stabilization through metal coordination. Scale bars are 10 μm for FIG. 20B, 4 μm for FIG. 20C, 1 μm for FIG. 20D, and 500 nm for FIG. 20E.

FIGS. 21A-21C are Raman microscopy images of S. cerevisiae-TiO₂ biohybrids. FIG. 21A is a schematic model of engineered S. cerevisiae-TiO₂ biohybrids. FIGS. 21B and 21C are mapping images generated from the Raman peaks at 530 cm⁻¹ and 1150 cm⁻¹ which correspond to the vibrations of Ti—O (TiO₂) and C—C (membrane).

FIGS. 22A and 22B show the Raman microscopy line scan of S. cerevisiae-TiO₂ biohybrid. FIG. 22A is a schematic showing position of Raman microscopy line scan on S. cerevisiae-TiO₂ biohybrid. FIG. 22B is a histogram of signals at 530 cm⁻¹ and 1150 cm⁻¹ which correspond to the vibrations of Ti—O (TiO₂) and C—C (membrane). The line scan confirmed the core-shell structure of assembled TiO₂ nanoparticles on the yeast cell surface.

FIGS. 23A-23C show the modular Assembly of E. coli-PS biohybrids. FIG. 23A is a schematic demonstration of E. coli-PS biohybrids assembly. FIGS. 23B and 23C are 3D fluorescence images of E. coli-PS biohybrids reconstructed from confocal fluorescence microscopy. The central area represented the E. coli cell was reconstructed based on blue DAPI signal while the surrounding PS particles were reconstructed based on the green fluorescence signal. Scale bars are 2 μm for FIG. 22B and 500 nm for FIG. 22C.

### DETAILED DESCRIPTION

In view of the limitations of the current systems as described herein, it would be highly desirable to develop strategies and biohybrid systems that are capable of harnessing and converting light energy into chemical energy in the form of electrons, cofactors and metabolite intermediates that are energetically expensive to generate or regenerate and closely intertwined with production pathways of useful metabolites and biomass. It would also be highly desirable that these biohybrid systems exhibit “plug-and-play” versatility that enables the pairing of any nanomaterials and biological systems in order to optimize the conditions for specific production pathways.

In a first aspect, the present disclosure provides biohybrid systems for absorbing and converting light energy into chemical energy and for photochemical biosynthesis. These hybrid systems are characterized by two distinct components: (i) functionalized photocatalytic inorganic nanoparticles and (ii) biological cells with modified cell surface membrane upon which the functionalized inorganic nanoparticles assemble. Notably, the hybrid systems of the present invention are also characterized by their modularity. Specifically, by virtue of the functionalization of both the inorganic nanoparticles and the biological cell surface, the modular platform of the present invention allows the versatility of a plethora of different types of nanoparticles to be combined with different types of cells.

As used herein the term “photochemical biosynthesis” refers to the production of compounds, molecules and metabolites within living organisms or cells that is initiated by the absorption of energy in the form of light (i.e., photon), which includes but is not limited to photosynthesis, where the energy of sunlight is converted into chemical energy by forming carbohydrates from atmospheric carbon dioxide and water and releasing molecular oxygen as a byproduct.

As used herein, a “biological” cell is a biological unit that consists of at least cytoplasm enclosed a membrane that forms a whole living unicellular organism or part of a multicellular organism. In some embodiments, the biological cell in the hybrid systems of the present invention is a “heterotrophic” cell, which is a cell of, or a cell from a heterotroph, which is an organism that cannot produce its own food but rely instead on the intake of nutrition from other sources of organic carbon. Examples of a heterotrophic cell include but are not limited to a yeast cell (e.g., Saccharomyces sp.), a non-autotrophic bacterial cell (i.e., non-photoautotrophic and non-chemoautotrophic), a mammalian cell, etc. Examples of a heterotrophic bacterial cell include but are not limited to Escherichia coli, Enterobacter aerogenes, Lactococcus sp., Lactobacillus sp., Bacillus sp., etc. In other embodiments, the biological cell in the hybrid systems of the present invention is an “autotrophic” cell, which is a cell of, or a cell from an autotroph, which is an organism that is capable of producing its own food, e.g., by oxidation of organic or inorganic electron donors in their environments (i.e., “chemotroph” or “chemoautotroph”) or by capturing photon in light (i.e., “phototroph” or “photoautotroph”). In some embodiments, a phototrophic cell is a “photosynthetic” cell where the chemical energy is synthesized from carbon dioxide and water. In such systems where the biological cell is an autotroph, the hybrid systems of the invention act to supplement the native autotrophic metabolic processes. In some embodiments, the biological cell is a heterotrophic cell that is engineered to be able to fix carbon dioxide to create an artificial photosynthetic system. In sum, the present invention is not limited by the type of biological cell used in the hybrid systems. In some embodiments, the biological cell in the hybrid systems of the present invention can be categorized as a “prokaryotic” cell (e.g., a bacterial cell, etc.) or a “eukaryotic” cell (e.g., an animal cell, a plant cell, a fungi cell, a protozoan cell, an algae cell, etc.)

As used herein, the term “inorganic” refers to a chemical entity that lacks carbon typically cannot be found in natural living organisms, which includes metals, semimetals, metalloids and semiconductors in their atomic, molecular and alloy forms

As used herein, “organic” refers to a chemical entity that contains at least one carbon atom, such as but not limited to organic polymers, all allotropes of carbon (e.g., carbon nanotubes, graphite, etc.), hydrocarbons, etc..

The terms “functionalization” and “functionalized” are used to refer to the process or the state of having new functions (including structural functional groups, chemical properties, physical properties) added to a material by “chemically modifying the surface” of the material. In the hybrid systems described herein, both components of the system, namely the nanoparticles and biological cells have been functionalized. A “functionalization agent” is a chemical substance that imparts the new functions to the surface of the material. In some embodiments, at least part of the functionalization agent is adsorbed onto the surface of the altered material.

Since inorganic nanoparticles are not typically incorporated in natural living organisms, the primary rationale behind functionalization of these particles is to enable the particles to assemble onto the biological cell in the hybrid systems of the invention. In one embodiment, the inorganic nanoparticles are functionalized with a phenolic compound, such as a polyphenol. In one embodiment, the polyphenol is tannic acid, polydopamine, resveratrol, ellagitannin, gallic acid, catechol, or a combination thereof. In general, any compound that matches the White-Bate-Smith-Swain-Haslam (WBSSH) definition as set forth in Bate-Smith et al., 1962, 58(371):95-173 (incorporated herein by reference in its entirety is considered a polyphenol.

In a particular embodiment, the inorganic nanoparticles of the hybrid systems described herein are functionalized with tannic acid. In one embodiment, the inorganic nanoparticles are functionalized by dispersing or suspending the unmodified nanoparticles in a solution at a concentration of about 0.01-10% w/v, or about 0.1-10% w/v, or about 0.5-5% w/v, or any other ranges or values that fall there within. Then, then the polyphenol or any other suitable functionalization agent is added to the nanoparticle solution to a final concentration of about 0.05-1.0 mM, or about 0.1-0.5 mM, or about 0.25 mM, or any other ranges or values that fall there within. In some embodiments, the functionalization with polyphenols forms a homogenous nanofilm coating on the nanoparticles. Notably, the functionalized nanoparticles in accordance with the present invention are prepared ex situ and prior to assembly on the biological cell surface.

The multidentate characteristic of the polyphenol enables multiple points of attachment of each polyphenol molecule to the biological cell surface and to each other. Each polyphenol molecule assembles at the cell surface through a mixture of hydrogen and hydrophobic interactions with the cell surface and with the adjacent polyphenol molecule(s). The optical density ratio of the functionalized nanoparticles over the cell, measured at 595-600 nm, is about 1.2-5.0 (i.e., about 1.2-5.0 nanoparticles per cell), or about 1.5-3.0, or about 1.6-2.0, or any other ranges or values that fall there within.

In some embodiments, the attachment of the functionalized nanoparticles to the cell surface is further strengthened by metal ion ligand coordination of the polyphenol molecules, which is achieved by addition of an equimolar amount (equal to the added polyphenol or any other functionalization agent) of a metal ion to the aforementioned nanoparticle solution, i.e., to a final concentration of about 0.05-1.0 mM, or about 0.1-0.5 mM, or about 0.25 mM, or any other ranges or values that fall there within. In general, any transition metal or noble metal without any high level of cytotoxicity can be used as a ligand between the polyphenol molecules. In one embodiment the metal ion ligand is selected from Ce³⁺, Al³⁺, Fe³⁺, Zn²⁺, Zr⁴⁺, and combinations thereof.

The negative surface charges of some cells, such as yeast and bacterial cells, can prevent the assembly of due to the strong electric repulsion, since the polyphenol functional groups also possess a net negative charge. Accordingly, in at least some embodiments, it is required that the surface membrane of the biological cell to be functionalized with a positive charge. In one embodiment, the positive charge is imparted through use of one or more cationic polymers as the functionalization agent. A non-exhaustive list of examples of cationic polymers include poly(allylamine) hydrochloride (PAH), poly(ethyleneimine) (PEI), poly-L-(lysine) (PLL), poly[2-(N,N-dimethylamino)ethyl methacrylate] (PDMAEMA), polyethylene glycol-PLL (PEG-PLL), PLL-g-dextran, polyamido amine (PAA), poly(amino-co-ester), poly(N-isopropylacrylamide (PNIPAM), cationic chitosan, cationic dextran, cationic cyclodextrin, cationic gelatin, cationic cellulose, a quaternary phosphonium cationic polymer, a quaternary ammonium cationic polymer, and copolymers thereof. In one embodiment, the cell surface is functionalized with poly(allylamine) hydrochloride (PAH). In some embodiments, common cationic polymers generally have amino groups on the side groups or the main chain, and the positive charge can be imparted by alkylation to form a quaternary ammonium salt, such as PEI, PDMAEMA, etc., but also have a pyridyl group and an imidazolium salt. In one embodiment, functionalization of the cells changes the zeta potential of cells changes from about −40 to about +40 mA. In one embodiment, the zeta potential of cells changes from about −30 to about +30 mA. In one embodiment, the zeta potential of cells changes from about −28 to about +20 mA.

For purposes of harnessing light energy and photochemical biosynthesis, nanoparticles with photocatalytic properties are assembled onto the cell surface. In some embodiments, the photocatalytic nanoparticles are semiconductor nanoparticles. In some embodiments, the photocatalytic nanoparticles are complex oxide nanoparticle, such as but not limited to those having the spinel structure (e.g., CoFe2O4, MnFe2O4, NiFe2O4) and perovskites (e.g., SrTiO3, BiFeO3, LaMnO3). In some embodiments, the photocatalytic nanoparticles are binary semiconductor nanoparticles. Non-limiting examples of suitable binary semiconductor materials include are silicon carbide (SiC), boron nitride (BN), boron phosphide (BP), aluminum nitride (AlN), aluminum phosphide (AlP), aluminum antimonide (AlSb), gallium nitride (GaN), gallium phosphide (GaP), gallium selenide (GaSe), gallium antimonide (GaSb), indium nitride (InN), indium phosphide (InP), indium antimonide (InSb), cadmium phosphide (Cd₃P₂), cadmium antimonide (Cd₃Sb₂), cadmium selenide (CdSe), cadmium sulfide (CdS), cadmium telluride (CdTe), zinc phosphide (Zn₃P₂), zinc antimonide (Zn₃Sb₂), zinc oxide (ZnO), zinc selenide (ZnSe), zinc sulfide (ZnS), zinc telluride (ZnTe), copper sulfide (Cu₂S), copper(I) oxide (Cu₂O), copper(II) oxide, tin sulfide (SnS), tin sulfide (SnS₂), tin telluride (SnTe), tin dioxide (SnO₂), bismuth telluride (Bi₂Te₃), bismuth trioxide (Bi₂O₃), bismuth iodide (BiI₃), bismuth sulfide (Bi₂S₃), titanium dioxide anatase (TiO₂), titanium dioxide rutile (TiO₂), titanium dioxide brookite (TiO₂), uranium dioxide (UO₂), uranium trioxide (UO₃), molybdenum disulfide (MoS₂), thallium bromide (TlBr), and combinations thereof. In some embodiments, the binary semiconductor materials are selected based on the specific metabolites to be synthesized and their respective pathway(s). In one embodiment, indium phosphide (InP) nanoparticles are assembled onto the cell surface.

The semiconductor nanoparticles may also be defined by their direct band gap values. In semiconductor physics, the band gap of a semiconductor is of two types, a direct band gap or an indirect band gap. The minimal-energy state in the conduction band and the maximal-energy state in the valence band are each characterized by a certain crystal momentum (k-vector) in the Brillouin zone. If the k-vectors are the same, it is called a “direct band gap”. If they are different, it is called an “indirect band gap”. The band gap is called “direct” if the crystal momentum of electrons and holes is the same in both the conduction band and the valence band; an electron can directly emit a photon. In an “indirect” gap, a photon cannot be emitted because the electron must pass through an intermediate state and transfer momentum to the crystal lattice Accordingly, the semiconductor nanoparticles used to assemble on the cell surface have a direct band gap of no higher than 2.0 eV, or about 1.0 eV to about 1.5 eV, or about 1.0 eV to about 1.5 eV, or any other ranges or values that fall there within.

In the light energy-converting and photochemical biosynthesis hybrid systems of the invention, the photoexcitation of the photocatalytic nanoparticles generates electrons, which are then harvested by the biological cell and used to generate redox cofactors such as but not limited to NADPH, NADH, and FADH. In one embodiment, the photo-generated electrons are used by the biological cell to generate NADPH.

In certain embodiments, the redox cofactor generated is further utilized to fuel one or more metabolic pathways that may be native or non-native for the production of metabolites. In one embodiment, the metabolic pathways utilizing the photo-generated NADPH are selected from any of the yeast metabolic pathways described in Suástegui et al. (J Ind Microbiol Biotechnol, 2016, 43(11):1611-1624), which is incorporated herein by reference in its entirety. In one embodiment, the yeast metabolic pathways utilizing the photo-generated NADPH are selected from the shikimic acid pathway, flavonoid pathway, stilbenoid pathway, and benzylisoquinoline alkaloid pathway. In some embodiments, the metabolic pathway utilizing the photochemically generated redox cofactor is an engineered metabolic pathway or a genetically modified pathway.

As used herein, a “native” metabolic pathway is one that is naturally present in a native or wild-type cell or organism. The definition of a “native” metabolic pathway as used herein extends to pathways that are naturally present in a native cell or organism but may be engineered to manipulate the expression levels of the part of or the entirety of the gene expression cassette in the pathway, such as by mutation of certain genes or alteration of the promoter controlling expression levels. On the contrary, a “non-native” metabolic pathway is one that is not naturally present in a native cell or organism, but is instead built or incorporated in the cell or organism by synthetic biology, metabolic engineering and pathway engineering means or the like. Accordingly, as used herein, an “engineered” metabolic pathway is a genetically modified pathway. In accordance with the definitions of the present disclosure, an “engineered” or a “genetically modified” metabolic pathway may be native or non-native, and is considered native if the all enzymes, cofactors and metabolites remain the same. As used herein, the term “metabolite” encompasses all reactants, cofactors, secondary metabolites and products of a metabolic pathway, and includes adenosine triphosphate (ATP) and its precursors. The focus of the present invention is production of metabolites that are useful as precursors and intermediates in the manufacturing of fuels, nutraceuticals, pharmaceuticals and cosmetics, etc.

In some embodiments, the metabolic pathway utilizing the photochemically generated redox cofactors is enhanced by genetic modification. In one embodiment, the metabolic pathway utilizing the photochemically generated redox cofactor is enhanced by overexpression of at least one gene to increase carbon flux for the production of one or more metabolites. These genes may be part of the metabolic pathway utilizing the photochemically generated redox cofactors, or a related upstream pathway thereof. In one particular embodiment, the cell in the hybrid system is a yeast cell and at least one of TKL1, RKI1, ADH1, PGK1, aro4, aro4_{K229L}, aro1, and aro1_{D290A} is overexpressed to increase the carbon flux into one or more downstream metabolic pathways, such as the shikimic acid pathway, flavonoid pathway, stilbenoid pathway, and/or benzylisoquinoline alkaloid pathway.

In some embodiments, the biological cell of the hybrid systems of the invention is further genetically modified to at least partially disrupted one or more metabolic pathways that serve as the natural main source(s) of the targeted redox cofactor. This is especially advantageous in situations where these natural metabolic pathways are energetically costly. In one embodiment, the biological cell in the hybrid system is a yeast cell and the targeted redox cofactor for photo-generation is NADPH. The pentose phosphate pathway which is the primary natural source of NADPH in yeast is at least partially disrupted so as to circumvent the expense of losing two moles of CO₂ with every mole of NADPH generated. In one embodiment, the pentose phosphate pathway is disrupted by mutation or deletion of the gene zwf1.

In another aspect, the present disclosure provides a biohybrid cell system not designed specifically for light energy conversion and photochemical biosynthesis. This system comprises a biological cell having a surface membrane that is chemically modified with a cationic polymer, a plurality of polyphenol-functionalized nanoparticles assembled on the chemically modified surface membrane. In some embodiments, cationic polymer is selected from poly(allylamine) hydrochloride (PAH), poly(ethyleneimine) (PEI), poly-L-(lysine) (PLL), poly[2-(N,N-dimethylamino)ethyl methacrylate] (PDMAEMA), polyethylene glycol-PLL (PEG-PLL), PLL-g-dextran, polyamido amine (PAA), poly(amino-co-ester), poly(N-isopropylacrylamide (PNIPAM), cationic chitosan, cationic dextran, cationic cyclodextrin, cationic gelatin, cationic cellulose, and copolymers thereof. In one particular embodiment, the cationic polymer is poly(allylamine) hydrochloride (PAH). In some embodiments, the polyphenol-functionalized nanoparticles are polymer nanoparticles (e.g., polystyrene nanoparticles), semiconductor nanoparticles, metallic nanoparticles, electromagnetic nanoparticles, magnetic nanoparticles (e.g., for purposes of DNA methylation), fluorescent nanoparticles (e.g., polystyrene nanoparticles, for purposes of labeling), radioactive nanoparticles (e.g. for purposes of labeling), energy conversion nanoparticles (e.g., upconversion nanoparticles), nanoparticles suitable for use in electronics (e.g., gold nanoparticles, semiconductor nanoparticles, carbon nanotubes (CNTs), or a combination thereof, and the density of the nanoparticles on the cell surface are as described above. The interactions between the functionalized nanoparticles and the functionalized cell surface and the ionic interparticle interactions are as described above. These hybrid systems can have potential therapeutic or other biomedical applications.

The present disclosure also provides methods associated with thebiohybrid cell systems described herein. In one aspect, a method of producing a metabolite by exposing the hybrid system of the invention to a light source or illuminating the hybrid system with a light source, e.g., sunlight, is provided. In one embodiment, the metabolite produced by this method is selected from shikimic acid, a flavonoid, a stilbenoid, a benzylisoquinoline alkaloid, and combinations thereof.

A method of converting light energy into chemical energy is also provided herein, whereby a biohybrid cell system of the invention is exposed to a light source, or illuminated with a light source, e.g., sunlight.

In a further aspect, the present disclosure relates to a method of preparing an biohybrid cell system of the invention. The method includes steps of: (i) preparing the functionalized photocatalytic nanoparticles by adding a functionalization agent to a solution comprising photocatalytic nanoparticles; (ii) chemically modifying the surface membrane of the cell; and (iii) mixing the functionalized photocatalytic nanoparticles and the cell. In some embodiments, the method further includes addition of a metal ion, as described above, to the solution comprising photocatalytic nanoparticles. In some embodiments, the method further includes increasing the pH of the solution comprising photocatalytic nanoparticles after addition of the functionalization agent.

In a yet further aspect, the present disclosure relates to a method of modifying a biological cell, The method includes steps of: (i) chemically modifying the surface membrane of the cell with a cationic polymer; and (ii) mixing the cell with polyphenol-functionalized nanoparticles; wherein the functionalized nanoparticles assemble on the chemically modified surface membrane. In some embodiments, the method further includes preparing the polyphenol-functionalized nanoparticles by adding the polyphenol to a solution comprising nanoparticles. In some embodiments, the method further includes addition of a metal ion, as described above, to the solution comprising photocatalytic nanoparticles. In some embodiments, the method further includes increasing the pH of the solution comprising photocatalytic nanoparticles after addition of the polyphenol.

### EXEMPLIFICATION

While a number of embodiments of this invention has been described in the following examples, it is apparent that these basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references with their respective supplementary materials, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Abbreviations

G6P, glucose-6-phosphate; F6P, fructose-6-phosphate; G3P, glyceraldehyde-3-phosphate; PEP, phosphoenolpyruvate; Glu-6P, gluconate 6-phosphate; 6PDG, 6-phospho-D-gluconate; Ri5P, ribulose-5-phosphate; X5P, xylulose-5-phosphate; R5P, ribose-5-phosphate; S7P, sedoheptulose-7-phosphate; E4P, erythrose-4-phosphate; DAHP, 3-deoxy-D-arabinoheptulosonate 7-phosphate; DHQ, dehydroquinoate, CO₂, carbon dioxide. HEX, hexokinase, PGI1, phosphoglucose isomerase; ZWF1, glucose-6-phosphate 1-dehydrogenase; SOL3/4, 6-phosphogluconolactonase, GND1/2, 6-phosphogluconate dehydrogenase; RPE1, ribulose-5-phosphate 3-epimerase; RKI1, ribose-5-phosphate ketol-isomerase; TAL1, transaldolase; TKL1, transketolase; ARO4K229L, feedback insensitive DAHP synthase; ARO1, pentafunctional aromatic enzyme; subunits—C: DHQ synthase, E: DHQ dehydratase, D: DHS dehydrogenase.

### Overview

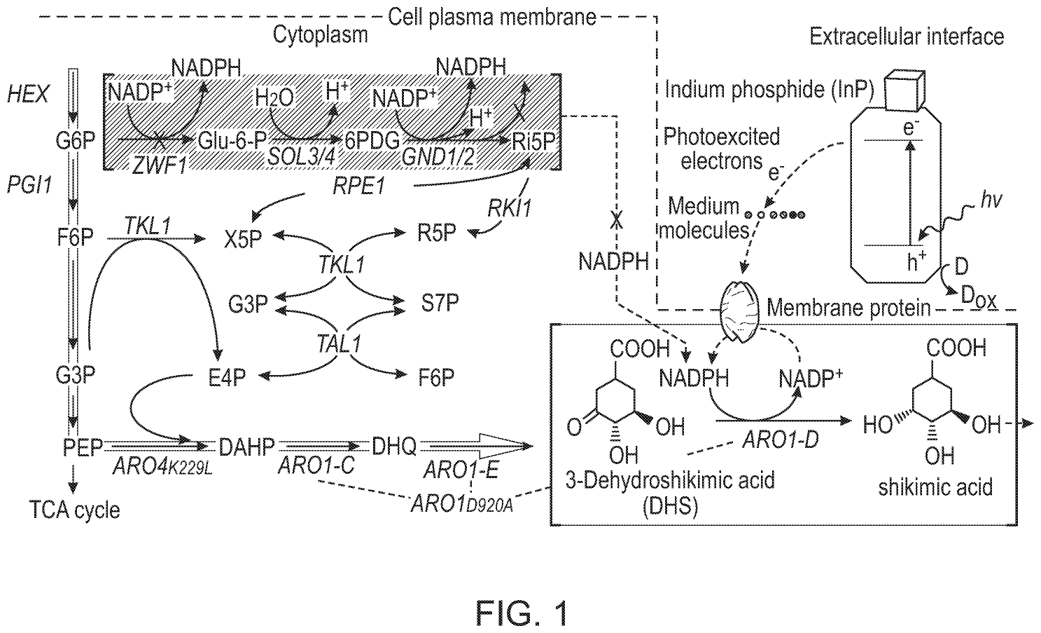

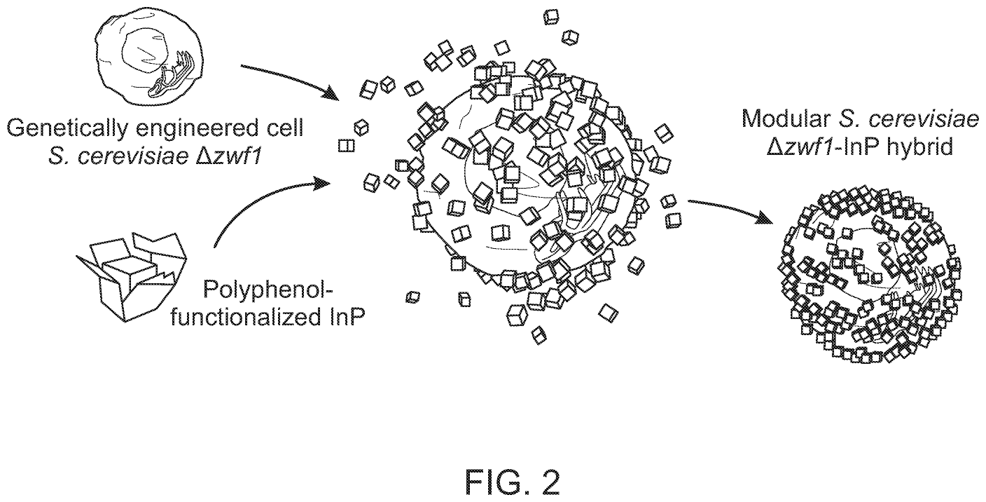

The following examples describe the preparation and characterization of a Saccharomyces cerevisiae-indium phosphide (InP) hybrid system, which combines rationally designed metabolic pathways and the electron donation capabilities of illuminated semiconductors (FIGS. 1 and 2).

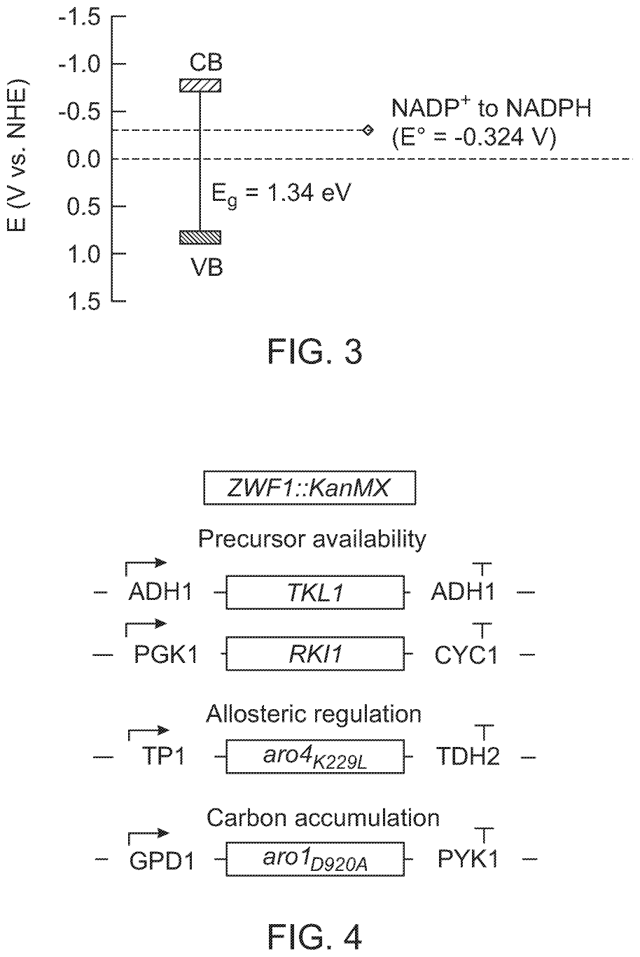

Indium phosphide (InP) served as a photosensitizer in this bioinorganic hybrid system, due to the appropriate direct band gap (Eg=1.34 eV) (FIG. 3). Additionally, its higher stability under oxygenic conditions and biocompatibility suggest that InP is an ideal material choice for the integration with biological systems. InP nanoparticles were synthesized independently and subsequently assembled on genetically engineered yeast cells by using a modified, biocompatible, polyphenol-based assembly method.

Yeast strain S. cerevisiae Δzwf1 was selected for the engineering of bioinorganic hybrid. Referring to FIG. 1, the deletion of the gene ZWF1, encoding the glucose-6-phosphate dehydrogenase enzyme, disrupts the oxidative portion of the pentose phosphate pathway. The null activity of this pathway causes the dramatic decrease of cytosolic NADPH generation, as indicated with the crosses in FIG. 1.

This exemplary bioinorganic system also enabled the study of the integrated function of the biohybrid system to regenerate NADPH which is closely linked with the biosynthesis of shikimic acid and aromatic amino acids. To this end and referring to FIGS. 1 and 4, S. cerevisiae Δzwf1 was further genetically engineered to overexpress four genes to enhance carbon flux through the shikimic acid pathway. The pentafunctional protein Aro1, which catalyzes the reduction of 3-dehydroshikimic acid (DHS) to shikimic acid, is highly selective for the cofactor NADPH. Hence, a low availability of cytosolic NADPH directly impacts the production of shikimic acid, leading to elevated accumulation of its precursor DHS. In the S. cerevisiae Δzwf1-InP hybrid system reducing equivalents can be generated from the illumination of surface-assembled InP nanoparticles, followed by the channeling of photoexcited electrons toward the generation of NADPH from NADP⁺. This NADPH can then fuel the ultimate conversion of DHS to shikimic acid. Therefore, the InP-S. cerevisiae Δzwf1 hybrid both enables us to evaluate the efficiency of NADPH regeneration and leads to the enhanced biosynthesis of a highly sought-after molecule through photon energy conversion.

### Part a—Preparation of Indium Phosphide Nanoparticles

### Example 1

### General Materials

Tannic acid (TA), iron(III) chloride hexahydrate (FeCl₃.6H2O), poly(allylamine hydrochloride) (PAH, Mw ˜17,500), tris(hydroxymethyl)aminomethane (Tris), 96% ethanol laboratory reagent, and phosphate-buffered saline (PBS) were purchased from Sigma-Aldrich (U.S.A). HPLC standards of 3-dehydroshikimic acid (DHS) and shikimic acid were purchased from Sigma-Aldrich (U.S.A). All of these materials were used as received. High-purity Milli-Q (MQ) water with a resistivity of 18.2 MΩ cm was obtained from an inline Millipore RiOs/Origin water purification system. All solutions were freshly prepared for immediate use in each experiment.

FluoSpheres polystyrene (PS) nanoparticles (40 nm) were purchased from Thermo Fisher Scientific (U.S.A.). The excitation wavelength is 505 nm and emission peak locates at 515 nm. Titanium(IV) oxide (TiO₂ 20 nm) nanopowders were purchased from Sigma-Aldrich (U.S.A.).

### Example 2

### Preparation of Indium Phosphide (InP) Nanoparticles

Indium(III) phosphide (InP) powers (pieces, 3-20 mesh, 99.998% trace metals basis, product number 366870) were purchased from Sigma-Aldrich (U.S.A.). InP nanoparticles were obtained through manual grinding. Briefly, ˜2.0 g of InP powders were weighted and transferred to a mortar and pestle porcelain set (Cole-Parmer, U.S.A.). The macroscopic InP powders were crushed to fine powders through gentle and consistent grinding process around 30 min. The grinded InP powders were transferred into a 1.7 mL tube (Eppendorf, U.S.A.). MQ water was used to suspend the grinded InP powders (0.5 2.5 mg) and sonication was applied to disperse the particles. InP particles were centrifuged at 8,000 g for 5 min to separate the sizes. The particles with larger sizes were spun on the bottom to form pellet while the smaller particles attached on the tube wall. The pellet was carefully discarded and the particles attached on the tube wall were resuspended by MQ water. To obtain InP with diameter smaller than 500 nm, the centrifugation-based separation process was repeated. Before the preparation of S. cerevisiae-InP biohybrids, the concentration of InP particles were measured by UV-Vis absorbance at 600 nm.

### Part B—Preparation of Shikimic Acid-Producing Engineered Yeast Strains

### Example 3

### Strains and Culture Media

The laboratory stains Saccharomyces cerevisiae BY4741 (MATa, his3Δ1, leu2Δ0, met15Δ0, ura3Δ0) and S. cerevisiae BY4741 zwf1Δ (MATa, his3Δ1, leu2Δ0, met15Δ0, ura3Δ0, zwf1Δ::KanMX) were used in this study (GE Dharmacon). The Yeast Extract-Peptone-Dextrose (YPD) medium was used for propagation of the cells before transformation of the shikimic acid plasmid. The YPD medium was composed of 10 g/L of yeast extract, 20 g/L of peptone, and 20 g/L of dextrose. Specifically, 6 g of yeast extract (BD Bacto Yeast Extract, BD Biosciences) and 12 g of peptone (BD Bacto Peptone Water Minimal Medium, BD Biosciences) were dissolved and stirred in 500 mL MQ. When preparing solid medium for plates, 10 g Agar (BD Bacto Agar, BD Biosciences) were added into the mixture. The mixture of solutions was autoclaved at 120° C. for 45 min, followed with addition of 100 mL 12% glucose after cooling down.

For constructing the shikimic acid producing strains, the plasmid pRS413-highAA was transformed into the S. cerevisiae following the quick and dirty transformation protocol with a few modifications. Briefly, a single colony was cultured overnight in YPD medium, and 50 μL of the saturated culture was aliquoted for transformation. The pelleted cells were resuspended in transformation mix composed of 200 μL 2 M lithium acetate, 800 μL 50% PEG 3350, 3 μL of 10 mg mL¹ salmon sperm, and 1 μg of plasmid. The mix was incubated at 37° C. for 30 min, washed twice with 200 μL of sterile water, and plated on selective solid medium.

The synthetic dropout medium lacking histidine (SC-His), used for culturing the S. cerevisiae strains harboring plasmid pRS413-highAA, consisted of 0.17% yeast nitrogen base without amino acids and without ammonium sulfate, 0.5% ammonium sulfate, complete supplement mix without histidine, and 2% glucose. Specifically, 1 g of yeast nitrogen base (Dico Yeast Nitrogen Base, BD Biosciences), 3 g of ammonium sulfate (Fisher BioRegents), and 0.96 g of yeast synthetic drop-out medium supplements without histidine (Sigma, U.S.A.) were dissolved and stirred in 500 mL MQ. When preparing solid medium for plates, 10 g Agar (BD Bacto Agar, BD Biosciences) were added into the mixture. The mixture of solutions was autoclaved at 120° C. for 45 min, followed with addition of 100 mL 12% glucose after cooling down.

Escherichia coli BL21 was used for the engineering of E. coli-PS biohybrids (Thermo Fisher Scientific, U.S.A.). Lysogeny broth (LB) medium was used for cell culturing.

### Example 4

### Genetic Construct

The genetic construct to enable the accumulation of DHS in the yeast strains harbored the overexpression of four genes under the control of strong constitutive promoters and cloned in the low copy-number plasmid, pRS413. The genes overexpressed to increase the carbon flux into the pathway were the transketolase (TKL1) and the ribose-5-phosphate ketol-isomerase (RKI1), under the control of the ADH1 promoter and PGK1 promoter, respectively. These two genes enhance the carbon flux through the pentose phosphate pathway leading to a higher availability of the rate limiting precursor erythrose-4-phosphate. To remove the feedback inhibition caused by the presence of the three aromatic amino acids in the media, the mutant gene aro4_{K229L} (DAHP synthase) was overexpressed under the control of the TP1 promoter. The leucine mutation to lysine in the position 229 deregulates an important effector-binding cavity, hence leading to a feedback insensitive enzyme capable of catalyzing the first committed step in the shikimic acid pathway even in the presence of tyrosine. Finally, the mutant pentafunctional aro1_{D290A} gene was overexpressed under the control of the GPD1 promoter. The alanine-to-aspartic acid substitution impairs the kinase subunit of the enzyme and prevents the conversion of shikimic acid to shikimate-3-phosphate.

### Example 5

### Polyphenol Functionalization of Indium Phosphide Nanoparticles, Cell Surface Modification, and Bioinorganic Hybrid Assembly

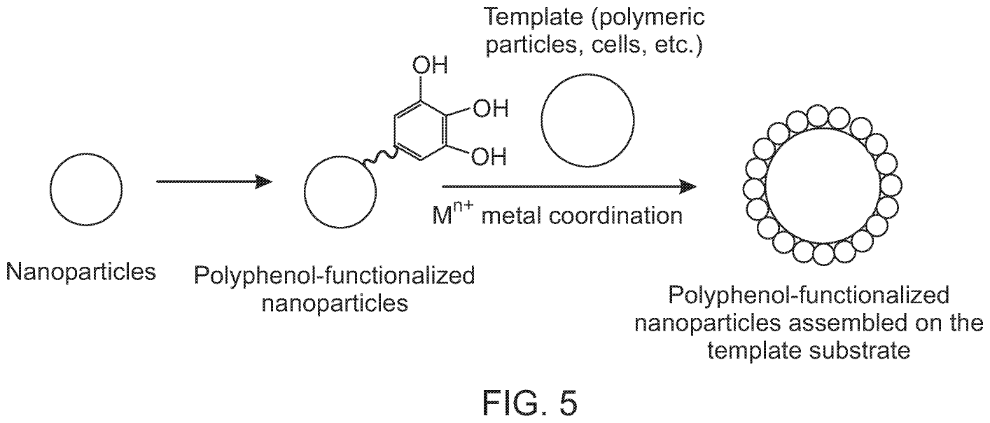

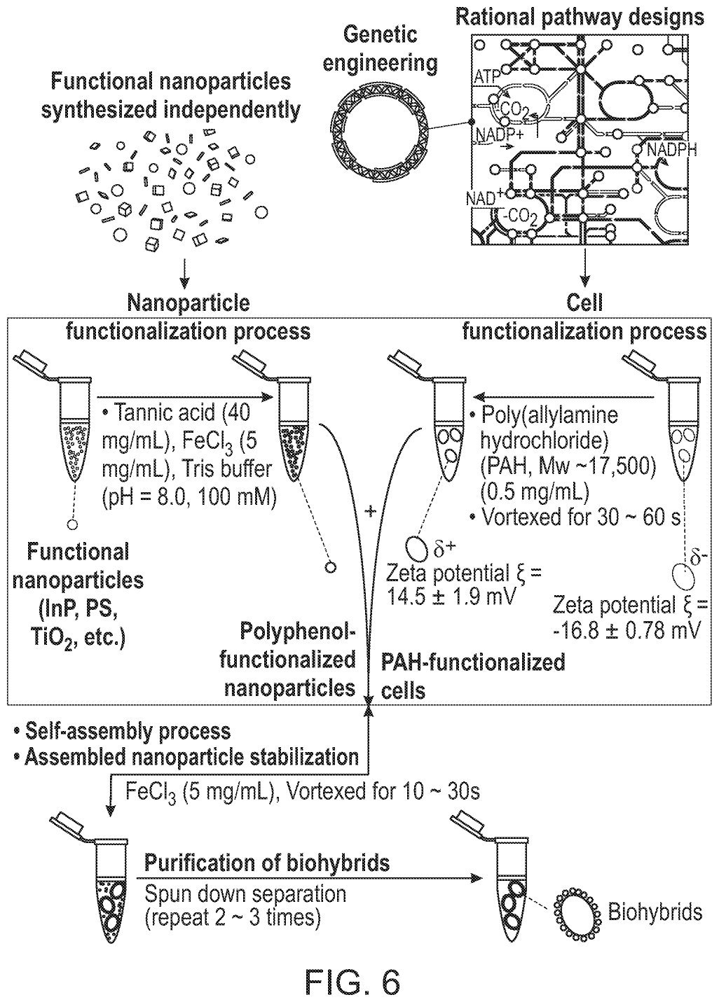

The assembly method of inorganic nanoparticles on cells were based on methods described in Guo et al. (Nature Nanotechnology, 2016, 11:1105-1111, see FIG. 5) but with modifications, as described in the following paragraphs and as illustrated in FIG. 6. Briefly, Guo et al.'s LEGO brick-inspired modularization of macroscopic building blocks comprises two steps: (i) particle surface functionalization with polyphenol moieties, which have been shown to be independent of the substrate and/or particle surface chemistry largely because of the multidentate properties of polyphenols; followed by (ii) particle assembly directed by interfacial molecular interactions (including hydrogen bonds and hydrophobic interactions) and interparticle locking of the functionalized particles using metal ions. All solutions were freshly prepared and filtered through 0.2 μm pores for immediate use. The standard preparation process is described as follows. The nanoparticles (InP, TiO₂, polystyrene PS, etc.) were suspended in MQ water (0.5-5% w/v).

### Polyphenol Functionalization of Indium Phosphide Nanoparticles

FeCl₃.6H₂O (5 mg/mL) and tannic acid (40 mg/mL) solutions were added to the inorganic nanoparticles suspension sequentially. Tris buffer solution (pH 8.0, 100 mM) was added to raise the solution pH. The polyphenol-functionalized inorganic nanoparticles were washed with MQ water 3-4 times, washed and incubated with 70% ethanol 10 min, and finally washed with MQ water 3-4 times. In the washing process, the particles were spun down by centrifugation and the supernatant was removed. Sonication was applied to disperse the particles in the suspension. The monodispersity of the particles was necessary for the following assembly process on cell surface.

### Modification of Yeast and Bacterial Cell Surface

The galloyl and catechol groups on the polyphenol-functionalized InP nanoparticles can from multiple interactions with cell surface, providing driving forces for the particle assembly on cells. However, the negative surface charges of yeast and bacterium cells prevent the assembly due to the strong electric repulsion, as shown in a previous study by the inventors using colloidal atomic force microscopy (AFM). Therefore, it is essential to functionalize the surface of cells with positive charges to enable polyphenol-based assembly process. Positive-charged polyallylamine hydrochloride (PAH) polymer was used to adsorb on the cell surface. The application of PAH polymer on cell surface or any other functionalization of the cell surface with positive charges had not been previously described in Guo et al. The detailed preparation process is described as follows and as illustrated in FIG. 6. Cells were washed with MQ three times to remove the broth medium and resuspended in 500 uL MQ. During the washing steps, the cells were spun down by 2,000 g for 2 min. Then, 50 uL PAH solution (5 mg/mL) was added into the 450 uL cell suspension (OD₆₀₀ 1.6˜3.2) and gently vertex for 20-40 s before spun down. The cells were washed with MQ three times to remove the excess PAH molecules. During the washing steps, the cells were spun down by 3,000 g for 2 min (1.7 mL eppendorf tube is recommended to use for washing process). Finally, the cells were resuspended in MQ and prepared for assembly with polyphenol-functionalized nanoparticles.

### Bioinorganic Hybrid Assembly

The assembly of inorganic nanoparticles on cell surface took place upon mixing of the nanoparticles with the modified cells. The concentrations of nanoparticles and cells were measured by optical density (OD) at 600 nm by NanoDrop 2000c spectrophotometers instrument (Thermo Fisher Scientific, U.S.A.). For the assembly of S. cerevisiae-InP biohybrids, the OD₆₀₀ of cells and InP nanoparticles were used as ˜1.0 and ˜2.0. The ratio of inorganic nanoparticle and cell numbers was varied based on different nanoparticles and cell types, and thus needed to be optimized to avoid particle aggregation. The number of InP should be more than that of cells, and the OD₆₀₀ ratio of InP to cells was generally 1.6-2.0. During the assembly process, the mixing suspension was vortexed for 10-60 s to facilitate collisions between the InP nanoparticles and S. cerevisiae cells. Then, the stabilization of particles on cells was achieved by adding additional metal ions to final concentration of 0.03 mg/mL FeCl₃ and an equal volume of PBS buffer solution (pH 7.4, 10 mM). S. cerevisiae-InP biohybrids were obtained after washing with MQ water for three times to remove the free InP nanoparticles. The centrifugation speeds used for S. cerevisiae-InP biohybrids were varied and optimized to avoid particle aggregation (2,000 g, 2 min).

### Example 6

### Modular Bioinorganic Hybrids with Different Nanoparticles

The modular method of polyphenol-based assembly method allows the design and engineering of biohybrids coupled with a wide range of inorganic nanoparticles (semiconductor, polymeric, organic particles, etc.). This enables the engineering of biohybrids potentially for a wider range of applications, from solar energy caption to cell surface engineering. The protocol of assembling different nanoparticles is as described above, while the centrifugation speeds used for functionalized particles were varied and optimized to avoid biohybrid aggregation. 40 nm polystyrene (PS) nanoparticles and 20 nm TiO₂ nanopowders were used as model nanoparticles to demonstrate the versatile choice of the modular platform. The functionalized PS nanoparticles were spun down at 8,000 g for 10 min. The functionalized TiO₂ nanopowders were spun down at 5,000 g for 2 min. The S. cerevisiae-PS nanoparticles biohybrids and S. cerevisiae-TiO2 were spun down at 2,000 g, 2 min.

### Example 7

### Modular Bioinorganic Hybrids with Different Prokaryotic and Eukaryotic Cells

The use of different microorganisms could access a wider catalog of desired metabolites due to the vast set of genetic tools and available knockout libraries. These advantages could allow the production of a selection of high-value added chemicals. Modular assembly method of biohybrids provides a platform for the use of a wide range of microorganisms and genetically engineered strains. The protocol of assembling different prokaryotic and eukaryotic cells is as described above, while the centrifugation speeds used for bacterium, yeast, or mammal cells were varied and optimized to avoid biohybrid aggregation. E. coli was chosen as model microorganism for the demonstration of versatility of the method. The E. coli cells were spun down by 4,000 g for 3 min.

### Example 8

### Colony Forming Unit (CFU) Assay

A 10 μL sample was taken at different time points throughout the fermentation experiments to measure colony forming units (CFU). The samples were diluted in sterile water at dilution rates ranging from 10⁻² to 10⁻⁵, and 50 μL aliquots were plated onto YPD solid medium. After two or three days of incubation at 30° C., the colonies were counted with an E-count colony counter pen (Heathrow Scientific, IL). The log₁₀ value of the total cell count (counted cells×dilution factor×50) was obtained and normalized to the zero-time point.

### Part C—Characterization of Nanoparticles, Cells, and Biohybrids

### Example 9

### Materials and Methods

### Instruments and Software

3D-reconstructed florescence microscopy imaging was performed using a Leica SP5X MP inverted confocal microscope equipped with a 60×1.42 NA oil immersion objective, with a set of standard filters for DAPI/CFP/FITC/AF488/AF568/Cy5/AF647. Image processing and 3D models were analyzed and generated with Imaris (Bitplane) software using the maximum intensity projection. Deconvolution images were taken on a series of z-sections within the top and bottom of a biohybrid. Scanning electron microscopy (SEM) images were obtained on a ZEISS FESEM Ultra-55 field-emission scanning electron microscope (Carl Zeiss, Germany), operating at an accelerating voltage of 5-10 kV. UV-Visible absorption and fluorescence measurements were conducted on an Infinite M200 PRO microplate reader (Tecan Group, Switzerland). Raman spectra and images were obtained using a Horiba multiline Raman spectrometer with the excitation source of 532 nm and 633 nm. It was equipped with an 800 mm spectrometer in 600 blaze grating and an 1800 blaze grating and a Synapse CCD detector (Horiba, Japan). Transmission electron microscopy (TEM) were performed on a JEOL JEM-1400 TEM instrument, operating at a voltage of 100 kV (JEOL USA, Inc.). Energy-dispersive X-ray spectroscopy (EDS) were performed on a JEOL 2010 FEG instrument (JEOL USA, Inc.), operating at a voltage of 200 kV. Particle zeta potential was measured by dynamic light scattering (DLS) on Malvern Zetasizer (Malvern, U.S.A.). HPLC spectra were collected from Agilent 1200 Series instrument and processed using Agilent ChemStation (Agilent, U.S.A). Ultrathin sections (about 80 nm) were cut on a Reichert Ultracut-S microtome, picked up on to copper grids stained with lead citrate and examined in a JEOL 1200EX Transmission electron microscope and images were recorded with an AMT 2 k CCD camera.

### SEM, TEM and Raman Microscopy Sample Preparation

2.0 μL of biohybrid suspensions in MQ water were allowed to air-dry on silicon wafers which were cleaned by acetone, ethanol, and MQ water for SEM and Raman microscopy. Formvar carbon-coated cupper grids were used to prepare TEM samples. The silicon wafers with dried samples were coated with platinum/palladium (80/20) using an EMS150T turbo-pumped sputter coater/carbon coater (EMS, U.S.A.).

### Ultrathin Section TEM Sample Preparation

S. cerevisiae Δzwf1-InP hybrids were centrifuged at 1,000 g for 2 min and the pellet was resuspended in 5 μL 20% BSA. The Yeast/BSA mixture was dispensed on the 100-μm side of a type A 6 mm Cu/Au carrier (Leica), covered with the flat side of a type B 6 mm Cu/Au carrier (Leica) and frozen in a high-pressure freezer (EM ICE, Leica). The samples were freeze substituted at −90° C. for 48 hours in an automated freeze substitution device (AFS2; Leica) in acetone containing 1% H₂O, 1% OsO4 and 0.1% uranyl acetate.

The temperature was increased 5° C. per hour up to 20° C. and the samples were rinsed several times in acetone at room temperature. The samples were infiltrated with Spurr's resin (EMS) mixed with acetone 1:1 overnight at 4° C. and moved to embedding molds filled with freshly mixed Spurr's resin at room temperature.

### Photochemical Production Designs

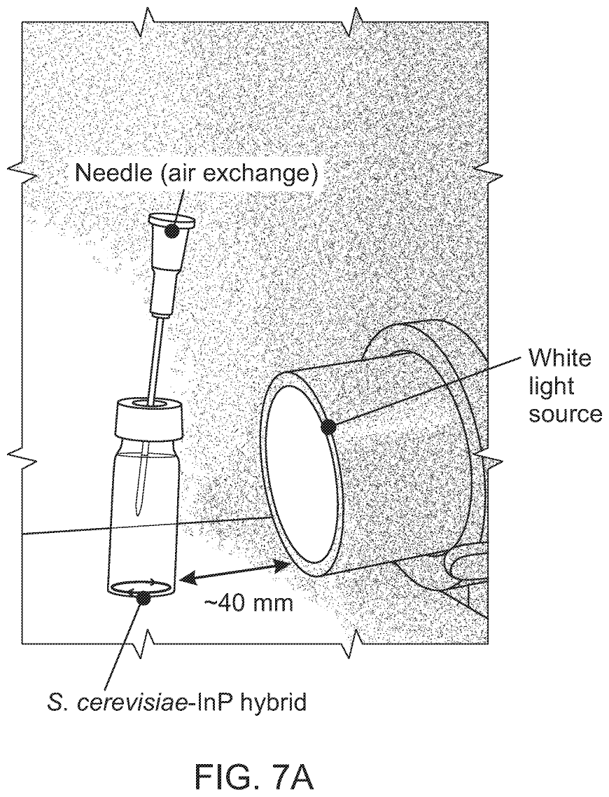

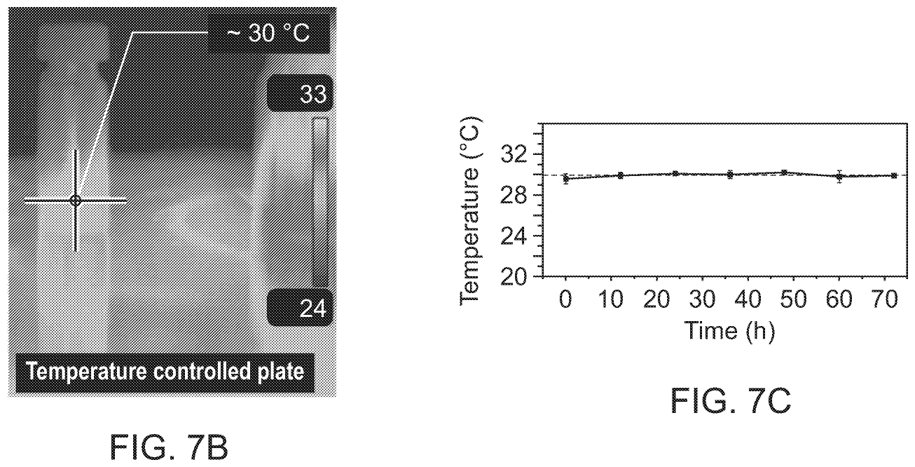

All photochemical production measurements were conducted with the biohybrid suspensions prepared as described herein and shown in FIGS. 7A-7C. Prior to photocatalytic experiments, 500 uL of biohybrid or cell suspensions were transferred to a transparent glass vial for consistent performance and standards (Agilent A-Line vials, U.S.A.). Each tube was stirred magnetically at 300 rpm and heated to a controlled temperature of 30° C. and tracked by using infrared camera (FLIR E75 Advanced Thermal Camera, U.S.A.). Specifically, a needle (Length: 40 mm, diameter: 1.2 mm, BD PrecisionGlide Needle) inserted into vial to enable air exchange. 500 μL suspension of S. cerevisiae-InP hybrids were added into the vials, stirred magnetically at 300 rpm and heated to a controlled temperature of 30° C. Illumination sources were employed by using a circular LED array composed of 20 high brightness violet LEDs with a broadband, cold-white output (Thorlabs, U.S.A.). The intensity of the LED arrays is 3.0 mW/cm² as measured from 100 mm away along the emission axis. The distance between vial and light source was fixed to ˜350 mm. According to the inverse-square law, the illumination intensity is 0.25 mW/cm² for the vials. The concentrations of photochemical productions were measured by high-performance liquid chromatography (HPLC) equipped with UV and IR detectors (Agilent 1200 Series, U.S.A). Spectra were processed using Agilent ChemStation.

### Quantification of Extracellular Metabolites

Samples were taken from the fermentation vials, and placed in 2 mL glass HPLC vial with a conical insert to quantify the production of shikimic acid, DHS, ethanol, glucose, and glycerol. The metabolites were analyzed by HPLC with a 1200 series stacked system from Agilent Technologies equipped with a diode array detector, a refractive index detector, and the Aminex HPX-87H column (300×7.8 mm) (Bio-Rad, Hercules, Calif.). The system was operated in isocratic mode using 5 mM sulfuric acid as mobile phase at a flow rate of 0.3 mL min⁻¹. Standard curves for each metabolites were constructed with pure standards. For shikimic acid, the retention time was observed at around 25 minutes with a maximum detection wavelength at 210 nm. The maximum peak for DHS was observed at around 32 minutes with a 235 nm wavelength. Glucose, glycerol, and ethanol were detected with the refractive index detector at 18 minutes, 26 minutes, and 40 minutes, respectively.

### Cytosolic-Free NADPH/NADP⁺ Ratio Calculations

The cytosolic-free NADPH/NADP⁺ ratio was calculated based on the following equilibrium reaction: DHS+NADPH+H⁺↔Shikimic acid+NADP⁺:

$$\frac{[ NADPH ]}{[ {NADP}^{+} ]} \times \frac{{H}^{+}}{{10}^{- 7}} = \frac{[ Shikimic acid ]}{{K}_{eq}^{′} \times [ DHS ]}$$

The value for the equilibrium constant, K′eq, was 9.0×10⁻⁵ was obtained from eQuilibrator.

### Example 10

### Characterization of Indium Phosphide Nanopowders

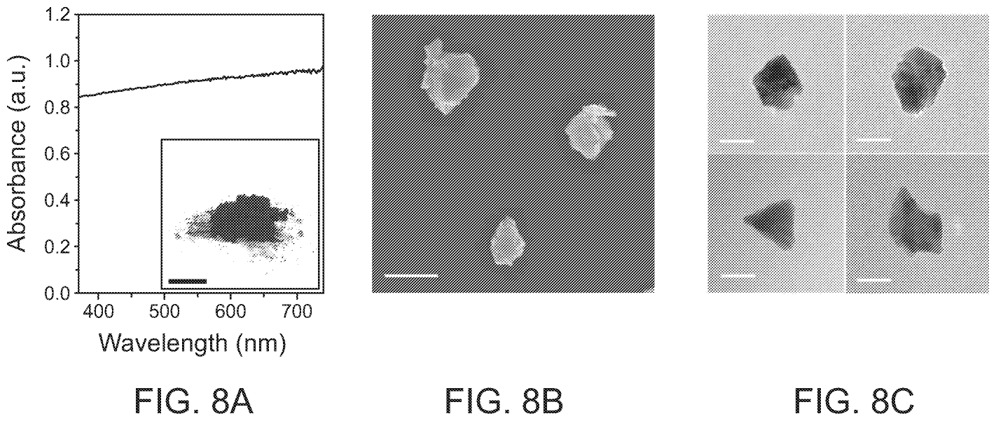

The indium phosphide (InP) nanoparticles used herein showed black color without significant absorption in visible wavelength (FIG. 8A). The morphology of the InP nanopowders after the grinding and separation process was analyzed using scanning electron microscopy (SEM) (FIG. 8B) and transmission electron microscopy (TEM) (FIG. 8C).

### Example 11

### Morphological Characterization of S. cerevisiae ΔZwf1-InP Biohybrid

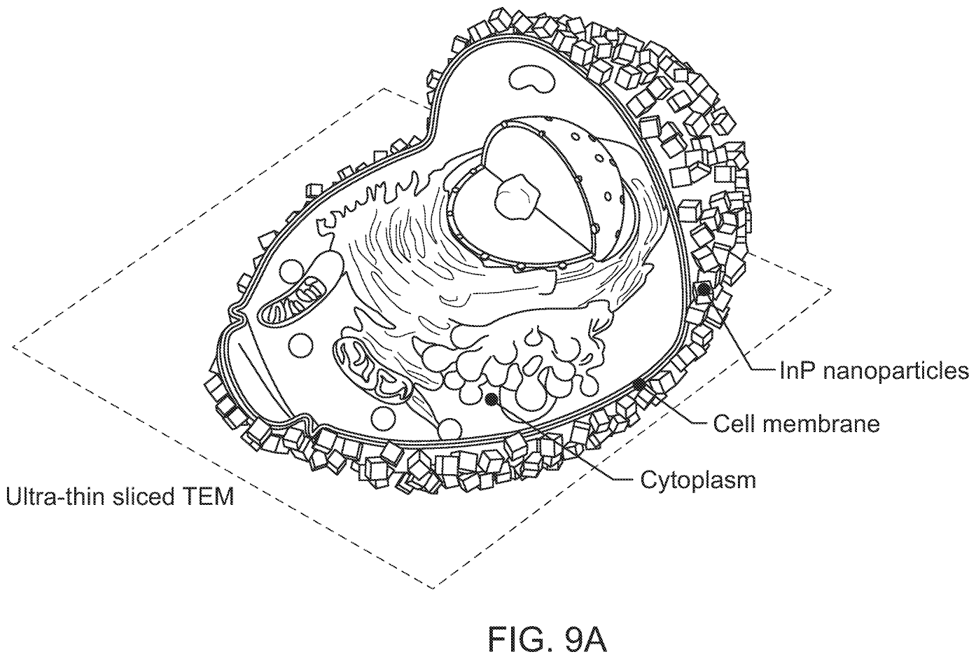

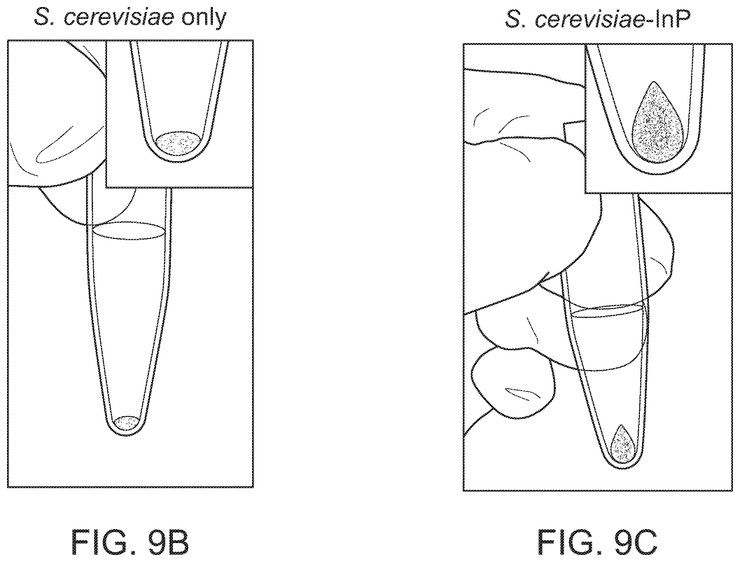

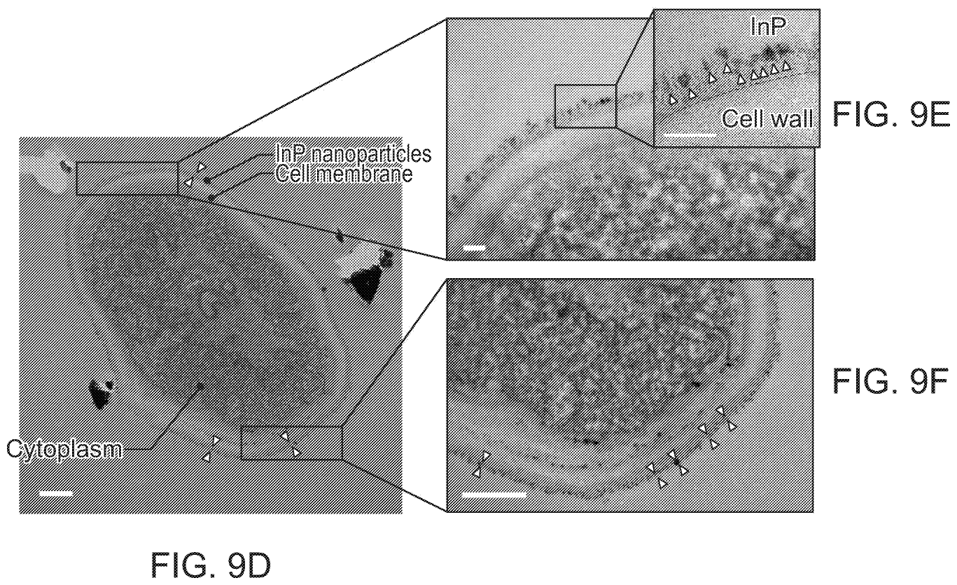

FIG. 9A is a schematic model of the S. cerevisiae Δzwf1-InP biohybrid. Photographs of centrifuged samples of bare cells (FIG. 9B) and S. cerevisiae Δzwf1-InP hybrids (FIG. 9C) in eppendorf tubes. The color change of pellet is ascribed to the assembly of InP nanoparticles on cells. The morphology of the S. cerevisiae Δzwf1-InP hybrid was examined using transmission electron microscopy (TEM) imaging of ultrathin cross-sectional specimen. The TEM images of FIGS. 9D-9F show the overall picture of S. cerevisiae Δzwf1-InP hybrid and assembled InP shell with darker contrast.

### Example 12

### Characterization of NADPH Regeneration and Shikimic Acid Production Pathways of S. cerevisiae ΔZwf1-InP Biohybrid

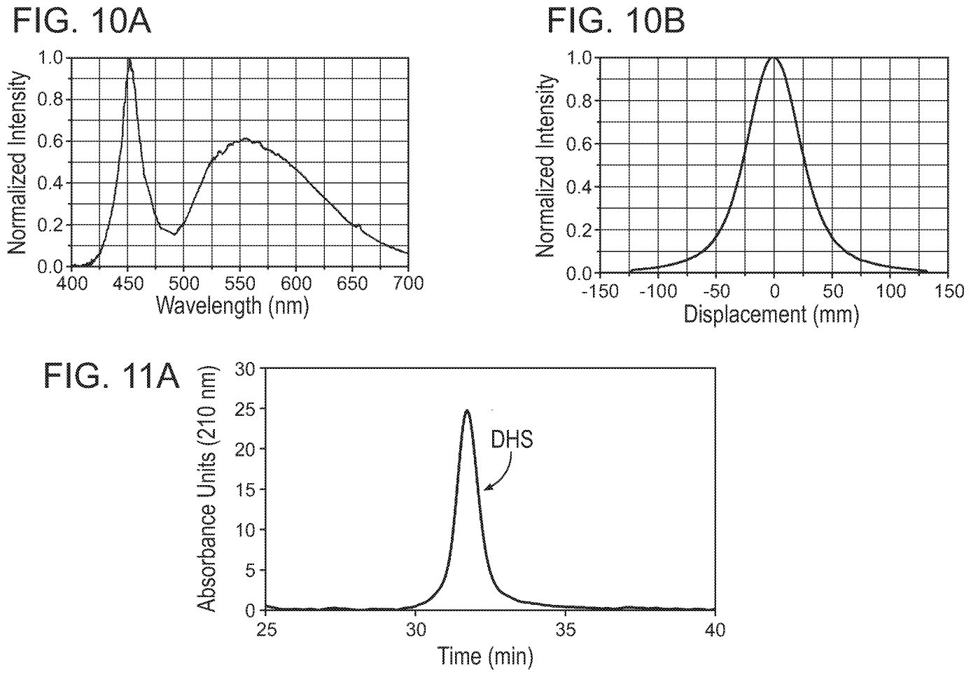

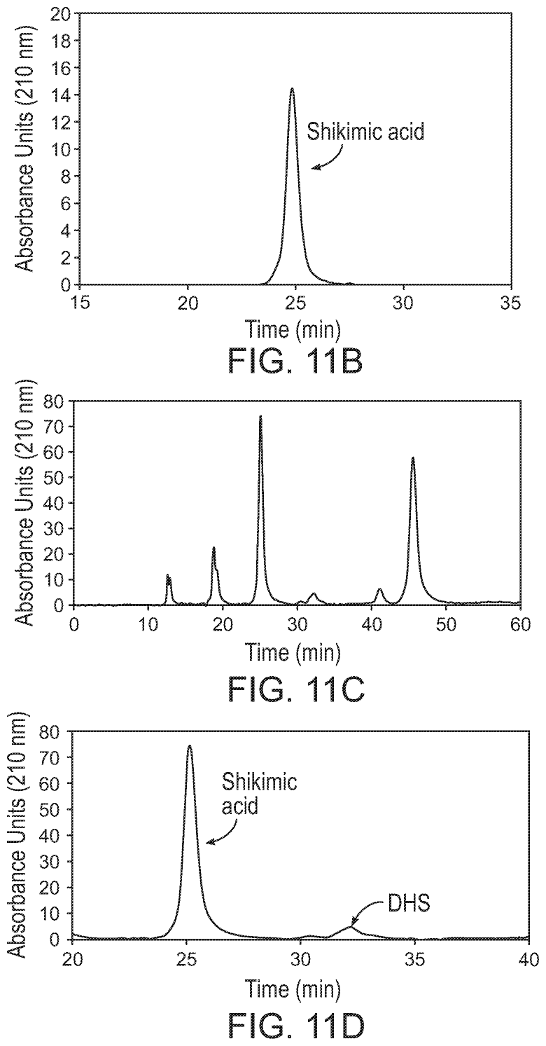

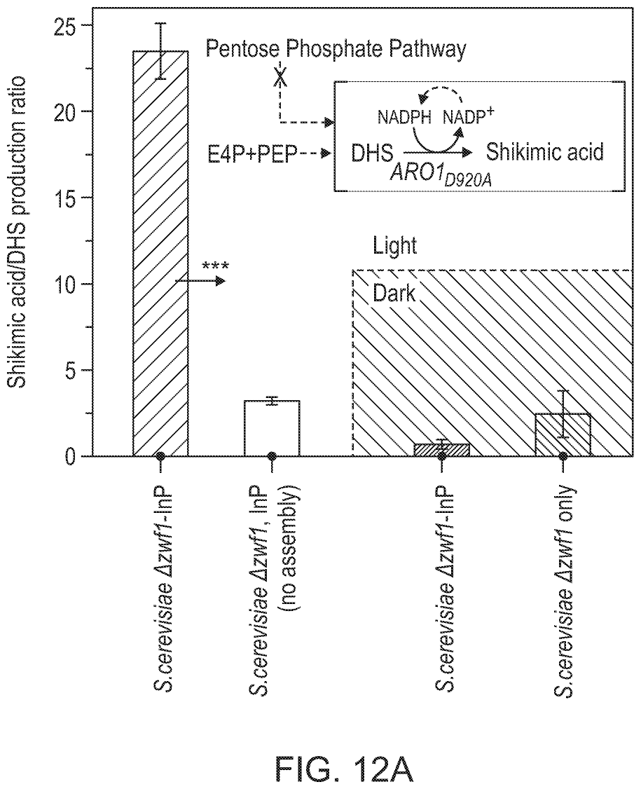

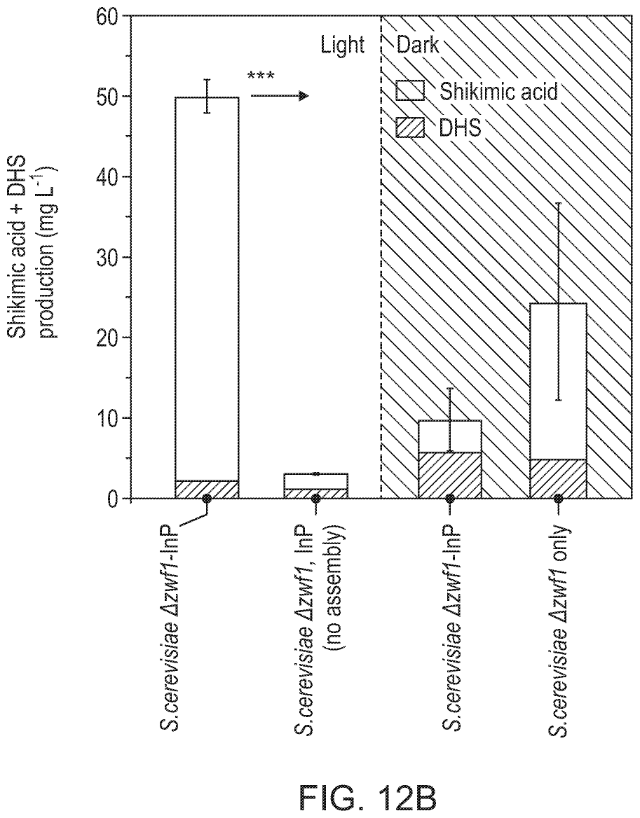

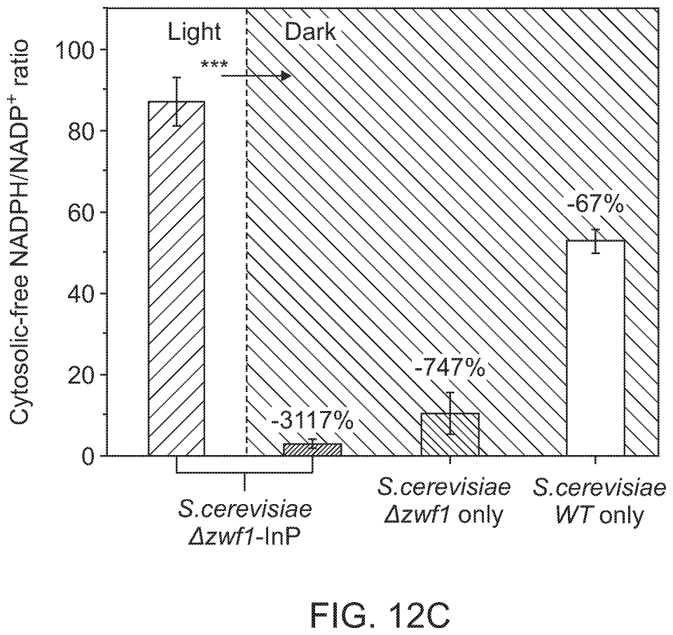

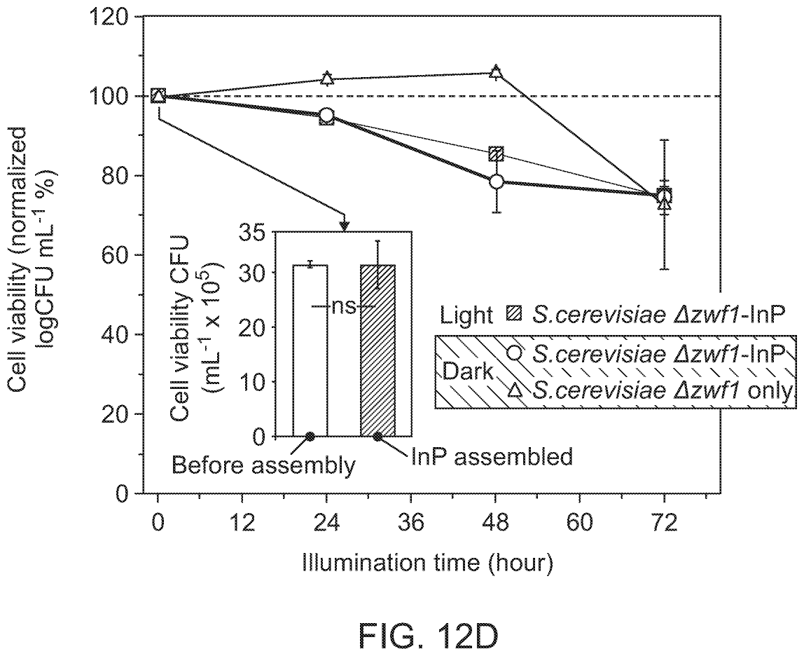

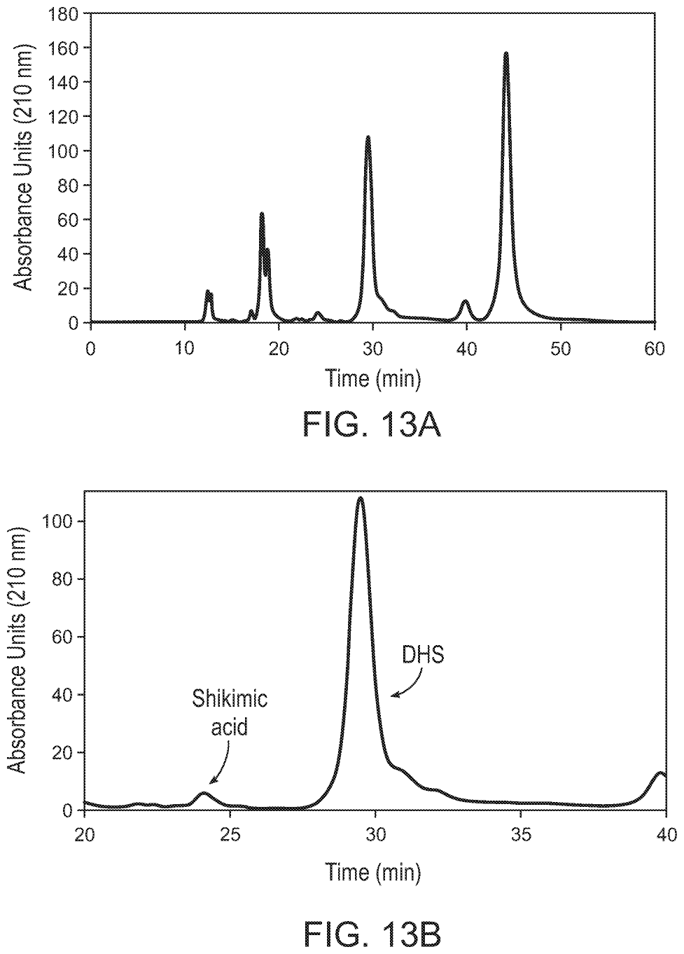

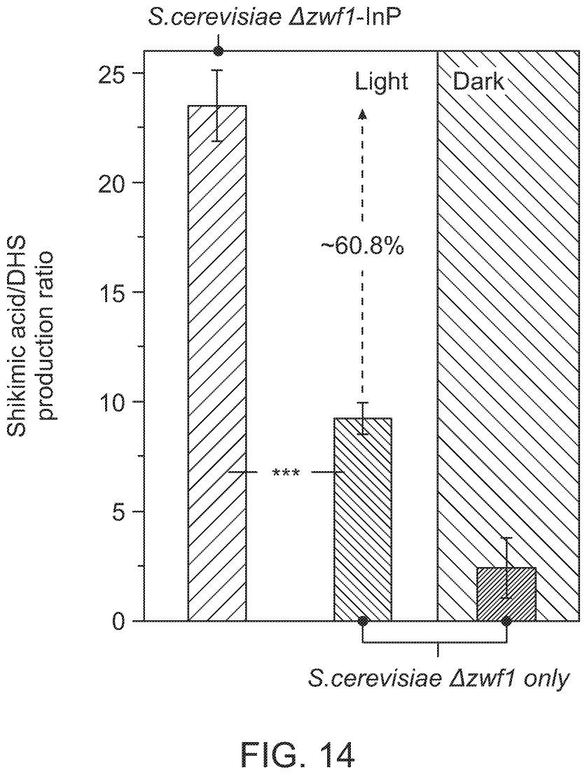

To verify the ability of the S. cerevisiae Δzwf1-InP biohybrid system to photochemically regenerate NADPH while maintaining high shikimic acid production, a series of control experiments, as described in Example 8 and shown in FIGS. 7A-7C, in which the presence of light and the InP nanoparticles was varied. After 72 hours of aerobic growth, S. cerevisiae Δzwf1-InP hybrids in the presence of illumination (0.25 mW/cm², FIGS. 10A and 10B) achieved the highest conversions, with a shikimic acid/DHS ratio of 23.5±1.6 (FIGS. 11A-11D; FIG. 12A). Similarly, a low shikimic acid ratio was observed in the presence of InP nanoparticles that were not assembled on the cell surface, suggesting the importance of proximity in enabling photochemical synthesis. S. cerevisiae Δzwf1 in the absence of InP also showed lower conversion efficiency (FIGS. 13A and 13B; FIG. 14). The total shikimic acid production of the illuminated biohybrid system was superior to all other conditions, with a final titer of 48.5±2.1 mg L⁻¹, showing an 11-fold increase compared to its counterpart with no illumination, and a 24-fold increase compared to engineered cells in the presence of InP without attaching on cell surface (FIG. 12B.

Shikimic acid/DHS ratio has previously been shown to serve as a metabolic readout for cytosolic levels of NADPH/NADP⁺. This facilitated calculation showed the highest NADPH/NADP+ ratio in the illuminated biohybrid experiment, reaching a value of 87.1 (FIG. 12C). Notably, this value was higher than even that measured for InP-free wild-type S. cerevisiae, which possesses fully functional machinery to produce NADPH through the oxidative PPP. S. cerevisiae Dzwf1 in darkness showed the lowest NADPH/NADP⁺ ratios regardless of the presence of InP. These results supported that the cell surface-attached InP enabled the photodriven regeneration of cofactor NADPH, facilitating the conversion of DHS to shikimic acid. Cell viability based on colony-forming units (CFU) assays showed no differences before and after the assembly of InP nanoparticles (FIG. 12D), confirming the biocompatibility of the particles assembly protocol. The insert in FIG. 12D shows the preparation of the bioinorganic hybrids does not affect initial CFU amount. After the first 24 hours of culture, cell count was lower for the biohybrids, regardless of the illumination scheme. Although the continuous illumination could also inhibit cell growth, this was not observed in the present studies, perhaps due to a protective effect from the assembled InP layer.

### Example 13

### Characterization of Glucose Consumption and Carbon Flux in S. cerevisiae ΔZwf1-InP Biohybrid

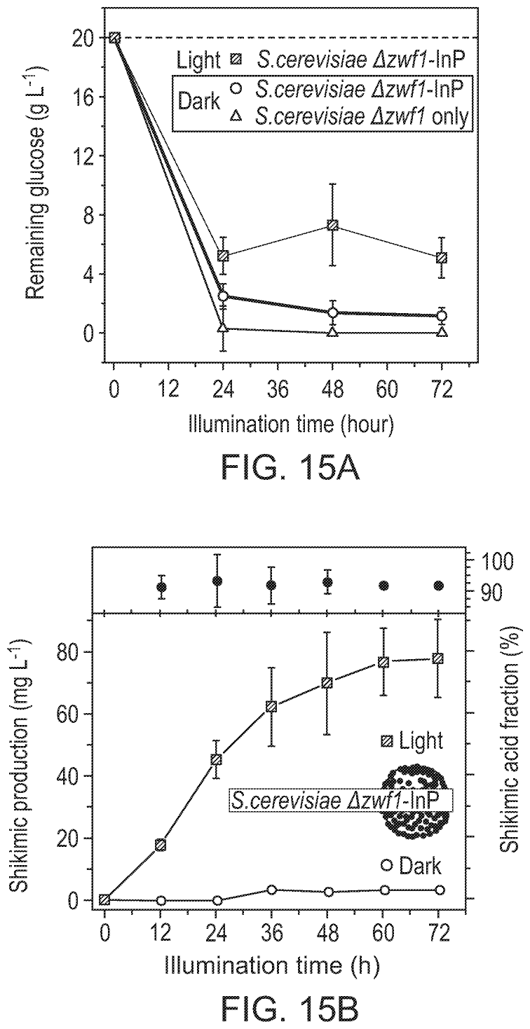

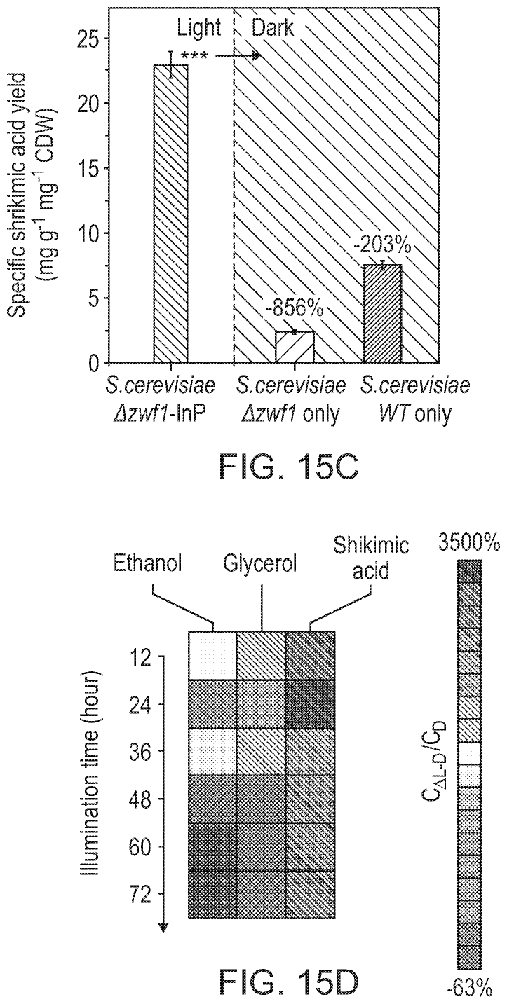

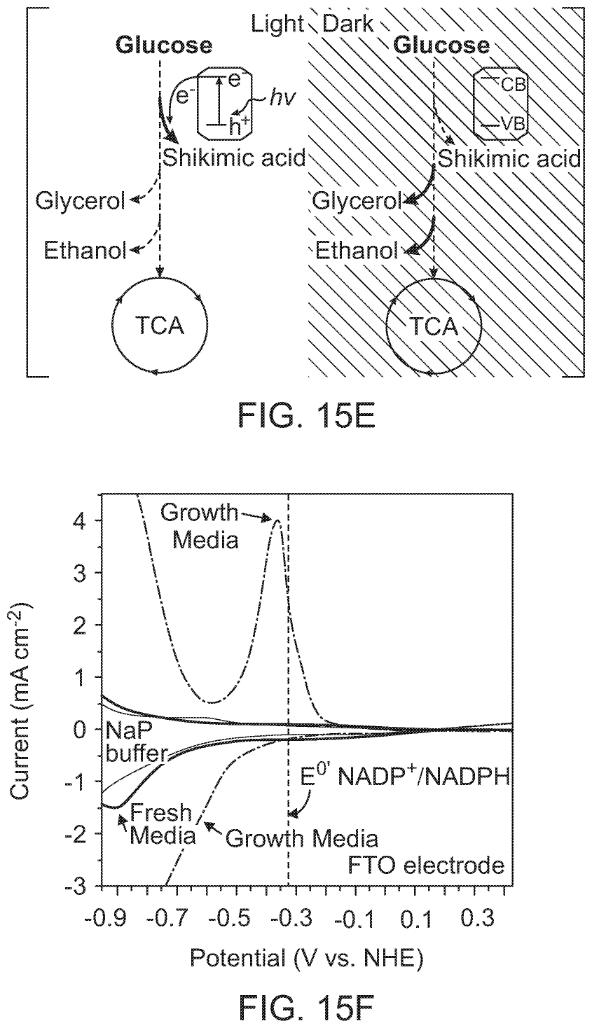

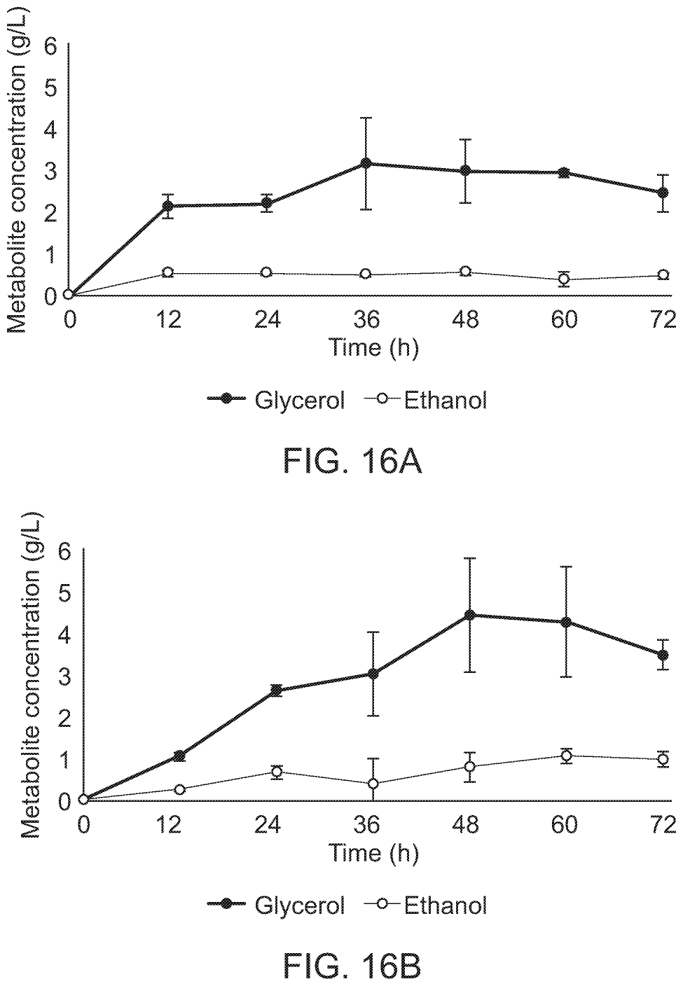

To further evaluate the metabolic performance of the biohybrid systems, the S. cerevisiae Δzwf1-InP biohybrid was characterized based on their ability to consume glucose and variations in carbon flux. Glucose was fully consumed by the bare cells during the first 24 hours, while nearly 25% of the total initial glucose remained unused in the complete biohybrid scheme (FIG. 15A). The shikimic acid production kinetics in the S. cerevisiae-InP hybrids showed that the conversion of DHS to shikimic acid occurred throughout the entire illumination period (FIG. 15B), suggesting a continuous supply of NADPH and potential accumulation of biosynthetic intermediates during the process. This was also supported by the consistent high mass fractions of shikimic acid (90%) through the entire experimental time frame. The specific shikimic acid yields of S. cerevisiae Δzwf1-InP hybrids surpassed those of the S. cerevisiae Δzwf1 and the wild-type bare cells cultured in darkness by 865% and 203%, respectively (FIG. 15C). To unravel the carbon flux variations caused by the photochemical activation in the central carbon metabolism of the hybrids, productions of secreted byproducts linked to other pathways, including ethanol and glycerol, were measured. (FIGS. 16A and 16B). FIG. 15D shows that the production of these byproducts by the illuminated biohybrids (CL) was lower than its counterpart under dark conditions (CD). This implied that the coupling of photoexcited InP could shunt S. cerevisiae Δzwf1 carbon primarily towards the desired shikimic acid synthesis, rather than it flowing toward alternative routes for NADPH regeneration (e.g., aldehyde dehydrogenase, Ald6) (FIG. 15E).

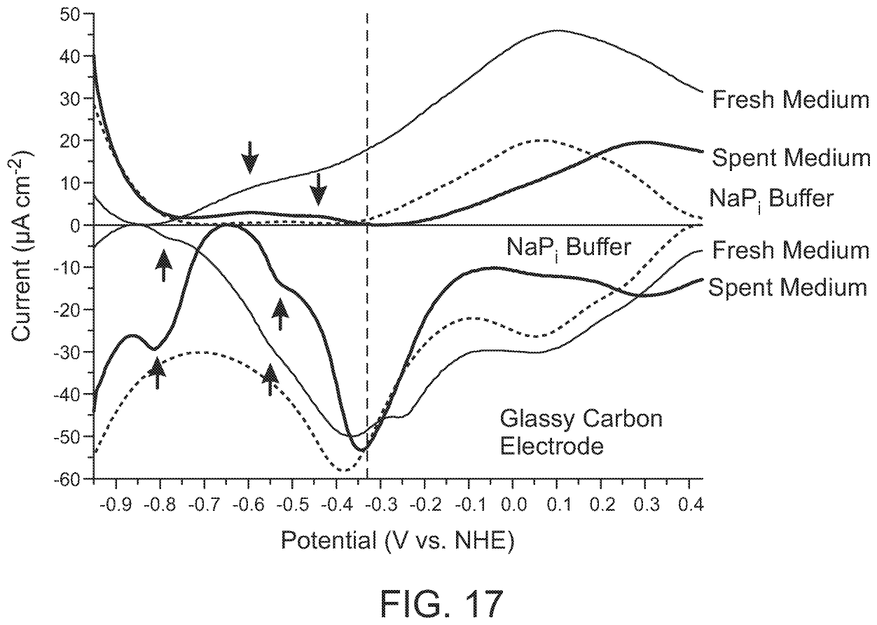

Differential pulse voltammetry of the growth medium identified the presence of such redox shuttles (FIG. 15C; FIG. 17). While the as-prepared medium exhibited DPV peaks more negative of the thermodynamic potential for NADP⁺/NADPH (E°=−0.324 V vs. NHE @ pH 7), a higher redox activity could be seen in the growth media after S. cerevisiae Δzwf1 growth. Without wishing to be bound by theory, the inventors suggest that this observation of heightened redox activity indicates the presence of a suitable redox mediator to shuttle electrons from InP. Alternatively, S. cerevisiae may utilize a system that is analogous to bacterial systems, which likely invokes a membrane-bound hydrogenase to make use of photochemically generated H₂.

### Example 14

### Modularity of Biohybrid Systems of the Invention

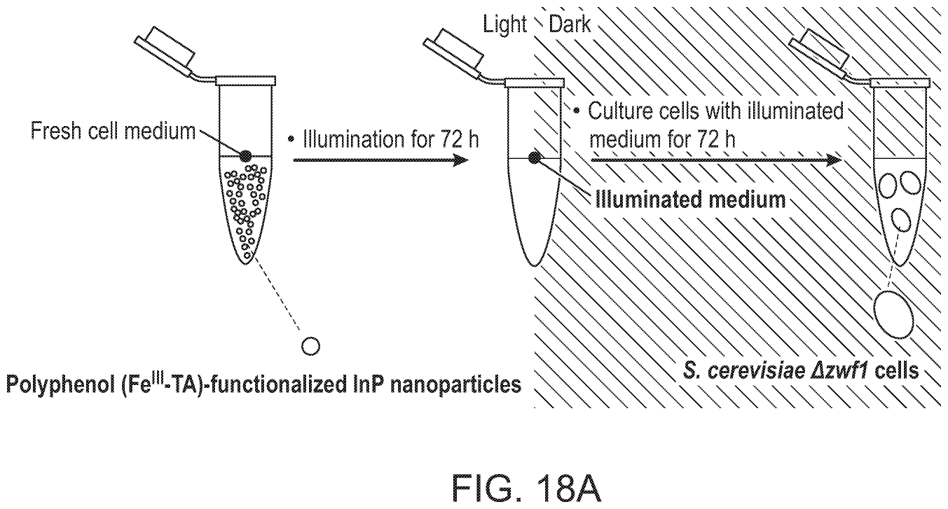

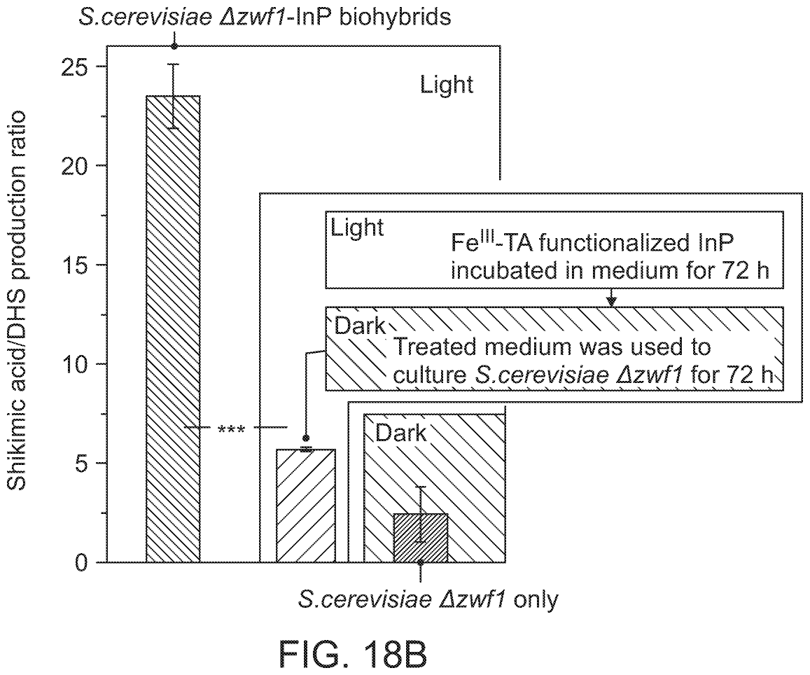

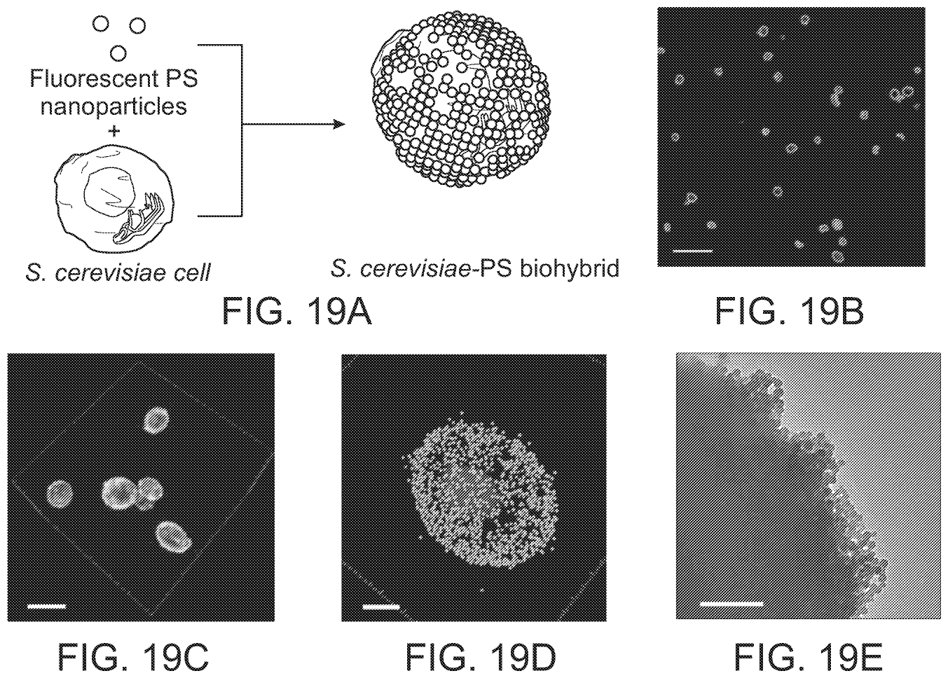

Examples of biohybrid systems with cell surface-coated nanoparticles have utilized a relatively narrow range of semiconductors and specific cell selections. This is, in part, because the nanoparticle synthesis was templated by specific chemical groups on the cell surface. The synthetic approach describe herein utilizes a polyphenol-based assembly method that could mediate a much broader range of cell-particle interactions. To illustrate this versatility, 3D-reconstructed fluorescence and TEM images are used to demonstrate the modularity of this synthetic approach with fluorescent polymeric and TiO₂ nanoparticles (FIGS. 18A and 18B; FIGS. 19A-19F).

FIGS. 18A and 18B illustrate the experiment conducted to determine the possible origins of electron transfer mediators. The schematic drawing in FIG. 18A depicts the experimental protocol in which growth medium is combined with polyphenol-functionalized InP nanoparticles and illuminated for 72 hours, then the nanoparticles are removed and the irradiated medium is used to culture the cells in darkness for 72 h. It was hypothesized that the irradiation of polyphenol-functionalized InP nanoparticles could possibly generate photochemical degradation products into the medium that could act as redox mediators or reactive oxidative species which could promote the increase of shikimic acid/DHS conversion and NADPH regeneration.

FIG. 18B shows that when polyphenol-functionalized InP nanoparticle irradiation and cell growth were performed as separate steps, the measured shikimic acid to DHS ratio was dramatically lower than for S. cerevisiae Δzwf1-InP hybrids in the light condition and similar to the values obtained for S. cerevisiae Δzwf1 cells only in darkness. These results negate the above hypothesis that degraded products from polyphenols (with Fe ions) or medium contribute to the observed results and supports the hypothesis suggested by the differential pulse voltammetry, that soluble redox mediators could be produced by the cells themselves during growth (FIG. 15D).

FIGS. 19A-19F illustrate the modular assembly of S. cerevisiae-polystyrene (PS) biohybrids. Green fluorescence PS was chosen as a model organic particle to demonstrate the modularity of the polyphenol based biohybrid assembly strategy. The fluorescence microscopy image of FIG. 19B reveals the coreshell structure of S. cerevisiae-PS biohybrids. Green represents the fluorescence of PS particles. Blue represents the nuclei of S. cerevisiae cells stained with DAPI. FIGS. 19C and 19D are reconstructed 3D images of S. cerevisiae-PS biohybrids from fluorescent confocal microscopy; while FIG. 19E is a TEM image of the cell surface of S. cerevisiae-PS biohybrids. FIGS. 19D and 19E show that PS particles were closely packed on the cell surface, suggesting strong interactions between polyphenol-based PS particles and yeast cell surface.

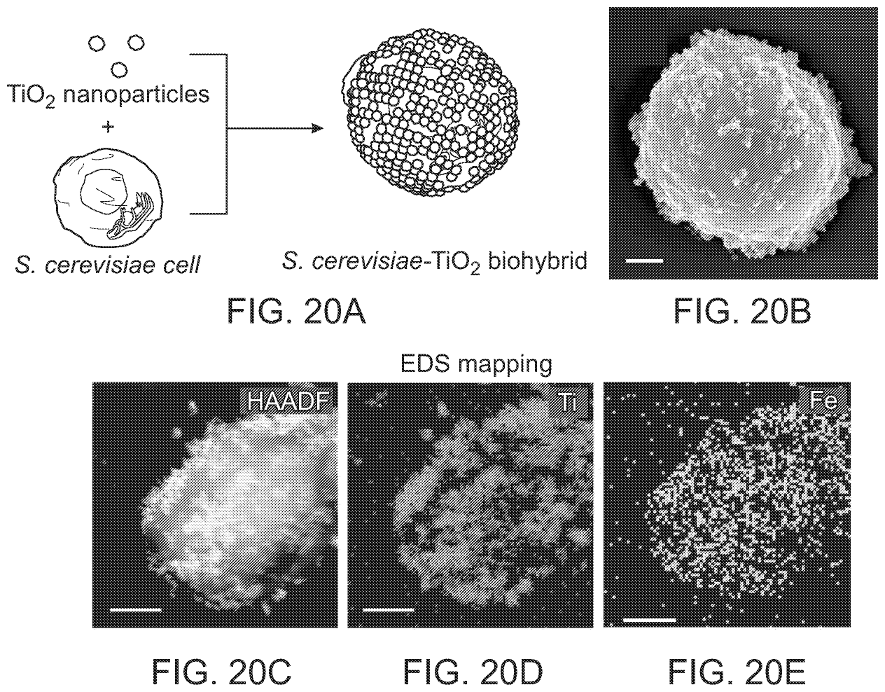

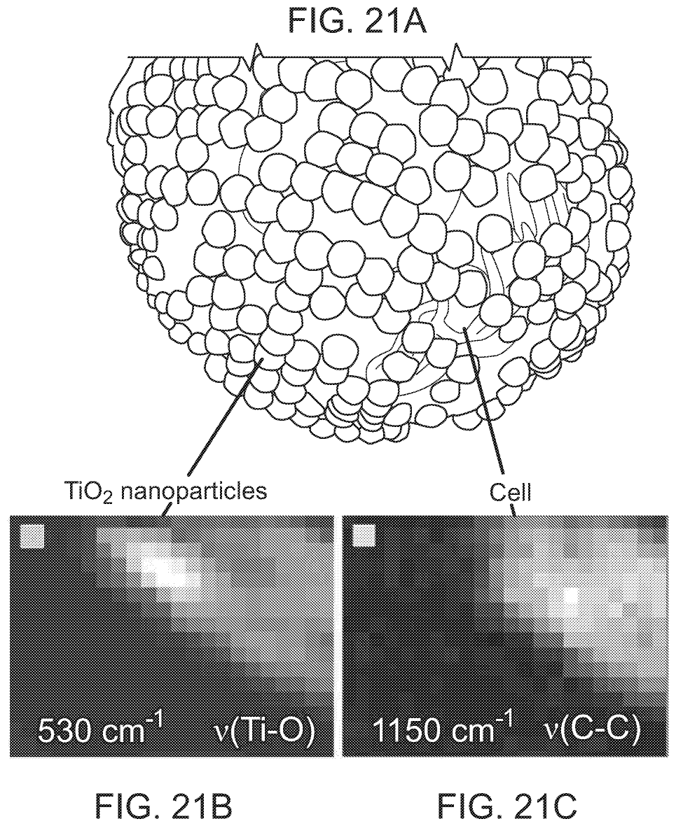

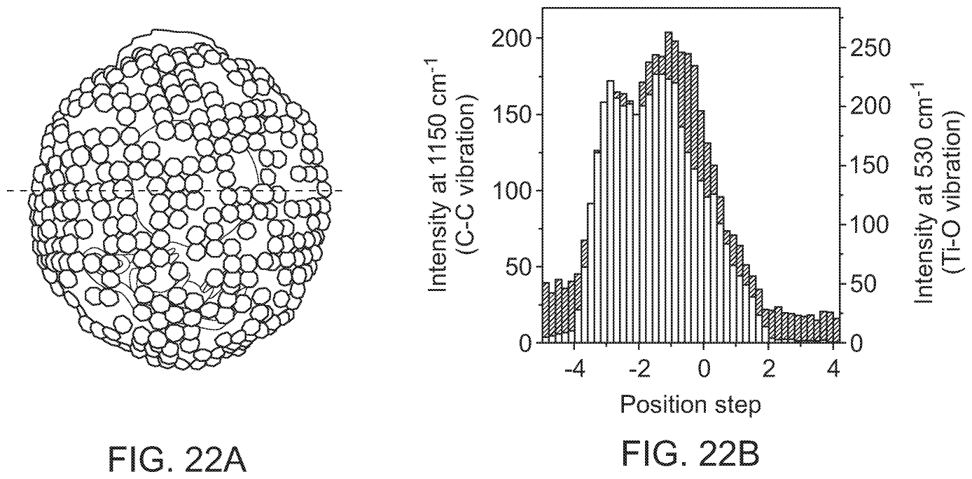

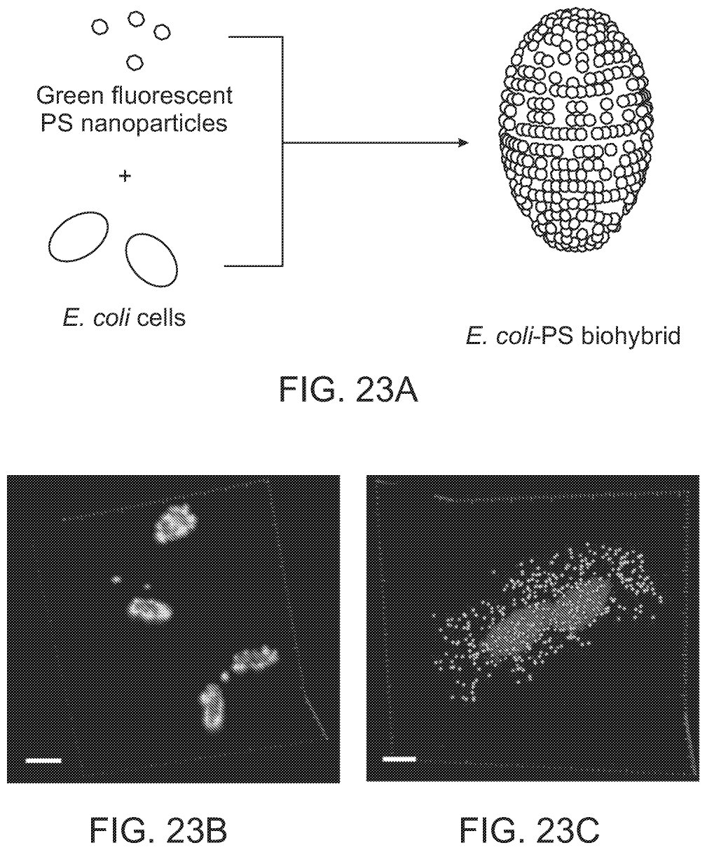

Raman spectral mapping and line scans confirmed the expected core-shell structure for the S. cerevisiae-TiO₂ hybrids (FIGS. 20A-20F; FIGS. 21A-21C). The modularity of the platform can also be applied to other cell types, enabling the creation of Escherichia coli-based bioinorganic hybrids (FIGS. 22A and 22B; FIGS. 23A-23C).

### Example 15

### Assembly of Indium Phosphide Nanoparticles Enhances Regeneration of Cytosolic NADPH and Oxidative Stress Effects on Biohybrid Performance

FIG. 14 shows that compared to the dark condition, illuminated, bare S. cerevisiae Δzwf1 cells did show a higher conversion ratio of DHS to shikimic acid, suggesting that other mechanisms, like oxidative stress response in cells, might contribute to the observed metabolic changes. Meanwhile, S. cerevisiae Δzwf1-InP hybrids achieved 60.8% higher ratio than S. cerevisiae Δzwf1 only in light. This significant enhancement highlights the advantage of assembled InP and supports the regeneration of cytosolic NADPH.

In addition to electron transfer from photoexcited InP nanoparticles to the cell, oxidative stress induced either directly by light, or as byproducts from InP irradiation, might modulate metabolic activity through altered genetic regulation. Light has been previously shown to activate oxidative stress pathways in yeast. InP quantum dots of several compositions have also been shown to generate varying amounts of reactive oxygen species. Therefore, activation of oxidative stress response pathways could cause the cells to redirect flux into other pathways that generate NADPH (e.g., aldehyde dehydrogenase, Ald6). The light alone (i.e. independent of InP) could explain the increase in shikimic acid/DHS ratio for the experiment where uncoated S. cerevisiae Δzwf1 cells were grown under illumination (FIG. 14). However, the yeast strain utilized herein to construct the biohybrid (S. cerevisiae Δzwf1) does not have the ability to generate NADPH via the pentose phosphate pathway. The PPP is the major upstream pathway capable of funneling carbon toward shikimic acid production, but it cannot be exploited in the present biohybrid because of this genetic knockout. The other pathways capable of generating NADPH are downstream of glycolysis, and would have to be balanced with flux through the shikimic acid pathway. In the illuminated biohybrid condition, both ethanol and glycerol—two fermentation byproducts that would be the direct result of increased flux through alternative NADPH-producing pathways—decreased compared to the corresponding non-illuminated case (FIGS. 16A and 16B). Finally, if oxidative stress arising from InP-generated reactive oxygen species was the primary explanation for the observed changes to cell metabolism in the illuminated biohybrid, then it would be likely that irradiated InP nanoparticles would have similar effects, regardless of their attachment to the cell surface. However, the present inventors observed a decrease in total shikimic acid and DHS production and no change in the SA/DHS ratio when S. cerevisiae Δzwf1 was grown in the presence of unattached InP with illumination.

In summary, the mechanism of metabolic modulation in illuminated S. cerevisiae Δzwf1-InP hybrids remains an active subject of investigation. The experimental results suggest that proximity of the InP particles to the cell surface is crucial for increasing the shikimic acid/DHS ratio and boosting the specific shikimic acid yields (FIGS. 12A and 15C).

### Example 16

### Conclusions

Unlike previously reported synthetic approaches of growing nanoparticles on cells based specific chemical groups, the approach undertaken herein (FIG. 6) allows for the independent synthesis and subsequent assembly of particles of arbitrary composition on genetically engineered cells. This separation between nanoparticle synthesis and cellular assembly can overcome the constraints of other biohybrid synthetic approaches, such as high temperatures, high pressures, and toxic precursors. This versatile approach streamlines the synthetic process and opens the door for broader choices for functional nanoparticles and cell types. The pathways of cells can be rationally designed through synthetic biology. The designed pathways (coupled with suitable inorganic materials) enable production of chemicals with higher carbon and energy efficiencies. The nanoparticles (i.e., InP, TiO₂, PS) can be functionalized through polyphenol-based coating. Polyphenols and metal ions form supramolecular networks on the surface of the nanoparticles. The surface charges of cells can be altered through adsorption of positive charged polymers (i.e., PAH). This is essential to facilitate polyphenol-based interfacial assembly between nanoparticles and cells. The assembly of nanoparticles on cells can also be triggered by adding Fe³⁺ ions.

The generation of redox cofactor NADPH in bioinorganic hybrid systems is of interest because of its central role in regulating enzymatic activity in many biosynthetic pathways. NADPH regeneration is energetically expensive and strongly intertwined with biomass production, and it is a common bottleneck in the production of metabolites through microbial cell factories. Although arduous strain engineering efforts have been focused on enabling a faster NADPH regeneration, these approaches, commonly accompanied by intricate pathway rewiring, inevitably introduce a strenuous burden on the cellular metabolism which prevents the system from reaching high-level production titers of the desired compounds. Moreover, as can be seen in FIG. 1, the primary source of NADPH in yeasts is the pentose phosphate pathway, which oxidizes a hexose sugar at the expense of losing two moles of CO₂, causing lower theoretical carbon yields. Therefore, it would be highly desirable to develop novel strategies aimed at decoupling NADPH generation from the central carbon metabolism, while maximizing carbon flux for the production of desired metabolites, which is what the present invention has achieved.

The production of shikimic acid by yeast-semiconductor biohybrids as a direct evaluation of photochemical cofactor regeneration sustained by illuminated semiconductor nanoparticles. Implementing this model in the yeast S. cerevisiae opens the door for a deeper understanding of the regeneration mechanism, and it allows for the expansion of this technique to the production of higher-value metabolites. For example, the production benzylisoquinoline alkaloids, which has already been established in yeast, requires the activity of more than ten membrane-bound cytochrome P450 oxidoreductases whose activity largely depends on the availability of NADPH as a primary electron donor. These reactions represent the bottlenecks of the pathway, limiting the production yields to the microgram per liter scale. It would be interesting to implement this technology presented in this work to elevate the production titers of the alkaloid family, as well as other potent drugs and nutraceuticals. Taking into account the multifarious availability of genetic tools, inorganic/organic nanoparticles, and cell types, this modular biohybrid platform is likely to enable profound new synthetic processes that will advance the biochemical production of a range of valuable and challenging targets.

While the applicant has described a number of embodiments of this invention, it is apparent that these basic examples may be altered to provide other embodiments that utilize the compounds and methods of this invention. Therefore, it will be appreciated that the scope of this invention is to be defined by the appended claims rather than by the specific embodiments that have been represented by way of example. Additionally, those skilled in the art will recognize, or be able to ascertain using no more than routine experimentation, many equivalents to the specific embodiments of the invention described herein. Such equivalents are intended to be encompassed by the following claims.

What is claimed is:

1. A biological hybrid cell system, comprising:
a biological cell having a chemically modified surface membrane; and
a plurality of functionalized photocatalytic nanoparticles assembled on the chemically modified surface membrane to enable the biological cell to absorb and convert light energy into chemical energy.

2. The cell system of claim 1, wherein the biological cell is a heterotrophic cell.

3. The cell system of claim 1, wherein the biological cell is a prokaryotic cell.

4. The cell system of claim 1, wherein the biological cell is a eukaryotic cell.

5. The cell system of claim 2, wherein the prokaryotic cell is a bacterial cell.

6. The cell system of claim 4, wherein the bacterial cell is an Escherichia coli cell.

7. The cell system of claim 3, wherein the eukaryotic cell is a yeast cell.

8. The cell system of claim 1, where the conversion of light energy into chemical energy produces one or more redox cofactors.

9. The cell system of claim 8, wherein the one or more redox cofactors comprise NADPH.

10. The cell system of claim 1, wherein the biological cell is genetically modified to comprise an enhanced a metabolic pathway that utilizes the chemical energy.

11. The cell system of claim 10, wherein the metabolic pathway is a native metabolic pathway.

12. The cell system of claim 10, wherein the metabolic pathway is a non-native, engineered metabolic pathway.

13. The cell system of claim 10, wherein the enhanced metabolic pathway utilizes NADP⁺/NADPH.

14. The cell system of claim 10, wherein the metabolic pathway is enhanced via overexpression of at least one gene to increase carbon flux for the production of one or more metabolites in the enhanced metabolic pathway.

15. The cell system of claim 14, wherein the biological cell is a yeast cell and the at least one gene that is overexpressed to increase the carbon flux is selected from TKL1, RKI1, ADH1, PGK1, aro4, aro4_{K229L}, aro1, and aro1_{D290A}.

16. The cell system of claim 10, wherein the enhanced metabolic pathway is selected from shikimic acid pathway, flavonoid pathway, stilbenoid pathway, benzylisoquinoline alkaloid pathway, and combinations thereof.

17. The cell system of claim 10, wherein the biological cell is further genetically modified to at least partially disrupt a native metabolic pathway that produces one or more redox cofactors.

18. The cell system of claim 17, wherein the one or more redox cofactors comprise NADP⁺/NADPH.

19. The cell system of claim 17, wherein the biological cell is a yeast cell and the disrupted native metabolic pathway is the pentose phosphate pathway.

20. The cell system of claim 18, wherein the pentose phosphate pathway is disrupted by mutation or deletion of the gene zwf1.

21. The cell system of claim 1, wherein the surface membrane is functionalized with a positive charge.

22. The cell system of claim 21, wherein the surface membrane is functionalized with a positive charge with a cationic polymer.

23. The cell system of claim 22, wherein the cationic polymer is adsorbed onto the surface membrane.

24. The cell system of claim 22, wherein the cationic polymer is selected from poly(allylamine) hydrochloride (PAH), poly(ethyleneimine) (PEI), poly-L-(lysine) (PLL), poly[2-(N,N-dimethylamino)ethyl methacrylate] (PDMAEMA), polyethylene glycol-PLL (PEG-PLL), PLL-g-dextran, polyamido amine (PAA), poly(amino-co-ester), poly(N-isopropylacrylamide (PNIPAM), cationic chitosan, cationic dextran, cationic cyclodextrin, cationic gelatin, cationic cellulose, a quaternary phosphonium cationic polymer, a quaternary ammonium cationic polymer, and copolymers thereof.

25. The cell system of claim 24, wherein the cationic polymer is poly(allylamine) hydrochloride (PAH).

26. The cell system of claim 1, wherein the photocatalytic nanoparticles are semiconductor nanoparticles.

27. The cell system of claim 18, where the semiconductor nanoparticles are binary semiconductor nanoparticles.

28. The cell system of claim 27, wherein the semiconductor nanoparticles are selected from silicon carbide (SiC), boron nitride (BN), boron phosphide (BP), aluminum nitride (AlN), aluminum phosphide (AlP), aluminum antimonide (AlSb), gallium nitride (GaN), gallium phosphide (GaP), gallium selenide (GaSe), gallium antimonide (GaSb), indium nitride (InN), indium phosphide (InP), indium antimonide (InSb), cadmium phosphide (Cd₃P₂), cadmium antimonide (Cd₃Sb₂), cadmium selenide (CdSe), cadmium sulfide (CdS), cadmium telluride (CdTe), zinc phosphide (Zn₃P₂), zinc antimonide (Zn₃Sb₂), zinc oxide (ZnO), zinc selenide (ZnSe), zinc sulfide (ZnS), zinc telluride (ZnTe), copper sulfide (Cu₂S), copper(I) oxide (Cu₂O), copper(II) oxide, tin sulfide (SnS), tin sulfide (SnS₂), tin telluride (SnTe), tin dioxide (SnO₂), bismuth telluride (Bi₂Te₃), bismuth trioxide (Bi₂O₃), bismuth iodide (BiI₃), bismuth sulfide (Bi₂S₃), titanium dioxide anatase (TiO₂), titanium dioxide rutile (TiO₂), titanium dioxide brookite (TiO₂), uranium dioxide (UO₂), uranium trioxide (UO₃), molybdenum disulfide (MoS₂), thallium bromide (TlBr), and combinations thereof.

29. The cell system of claim 28, wherein the semiconductors nanoparticles comprise indium phosphide (InP) nanoparticles.

30. The cell system of claim 18, where the semiconductor nanoparticles are complex oxide semiconductor nanoparticles.

31. The cell system of claim 30, wherein the complex oxide semiconductor nanoparticles are selected from complex oxide semiconductor nanoparticles having a spinel structure and perovskites.

32. The cell system of claim 26, wherein the semiconductor nanoparticles have a direct band gap of no higher than 2.0 eV.

33. The cell system of claim 32, wherein the semiconductor nanoparticles have a direct band gap of about 1.0 eV to about 2.0 eV.

34. The cell system of claim 33, wherein the semiconductor nanoparticles have a direct band gap of about 1.0 eV to about 1.5 eV.

35. The cell system of claim 1, wherein the photocatalytic nanoparticles are functionalized with one or more phenolic compounds.

36. The cell system of claim 35, wherein the photocatalytic nanoparticles are functionalized with a polyphenol.

37. The cell system of claim 36, wherein the polyphenol is selected from tannic acid, polydopamine, resveratrol, ellagitannin, gallic acid, catechol and combinations thereof.

38. The cell system of claim 1, wherein the plurality of functionalized photocatalytic nanoparticles forms a combination of hydrogen and hydrophobic interactions with the chemically modified surface membrane.

39. The cell system of claim 1, wherein the plurality of functionalized photocatalytic nanoparticles forms interparticle interactions with one another via metal ion ligand coordination.

40. The cell system of claim 39, wherein the metal ion ligand is selected from Ce³⁺, Al³⁺, Fe³⁺, Zn²⁺, Zr⁴⁺, and combinations thereof.

41. The cell system of claim 1, wherein the optical density ratio of the plurality of functionalized photocatalytic nanoparticles over the biological cell at 595-600 nm is about 1.2-5.0.

42. The cell system of claim 1, wherein the optical density ratio of the plurality of functionalized photocatalytic nanoparticles over the biological cell at 595-600 nm is about 1.5-3.0.

43. The cell system of claim 1, wherein the optical density ratio of the plurality of functionalized photocatalytic nanoparticles over the biological cell at 595-600 nm is about 1.6-2.0.

44. A biological hybrid photochemical biosynthesis system, comprising:
a biological cell having a chemically modified surface membrane, wherein the biological cell comprises:
an engineered metabolic pathway that utilizes the chemical energy to produce one or more metabolites;
a plurality of functionalized photocatalytic nanoparticles assembled on the chemically modified surface membrane to enable the biological cell to absorb and convert light energy into chemical energy.

45. The photochemical biosynthesis system of claim 44, wherein the biological cell is a bacterial cell or a yeast cell.

46. The photochemical biosynthesis system of claim 44, where the chemical energy is generated in the form of one or more redox cofactors.

47. The photochemical biosynthesis system of claim 46, wherein the one or more redox cofactors comprise NADPH.

48. The photochemical biosynthesis system of claim 44, wherein the biological cell is genetically modified to enhance carbon flux for the production of one or more metabolites in the engineered metabolic pathway.

49. The photochemical biosynthesis of claim 48, wherein the carbon flux is enhanced via overexpression of at least one gene that increases the carbon flux.

50. The photochemical biosynthesis system of claim 49, wherein the biological cell is a yeast cell and the at least one gene that is overexpressed to increase the carbon flux is selected from TKL1, RKI1, ADH1, PGK1, aro4, aro4_{K229L}, aro1, and aro1_{D290A}.

51. The photochemical biosynthesis system of claim 44, wherein the engineered metabolic pathway is selected from shikimic acid pathway, flavonoid pathway, stilbenoid pathway, benzylisoquinoline alkaloid pathway, and combinations thereof.

52. The photochemical biosynthesis system of claim 48, wherein the biological cell is further genetically modified to at least partially disrupt a native metabolic pathway that produces NADP⁺/NADPH.

53. The photochemical biosynthesis system of claim 52, wherein the biological cell is a yeast cell and the disrupted native metabolic pathway is the pentose phosphate pathway.

54. The photochemical biosynthesis system of claim 53, wherein the pentose phosphate pathway is disrupted by mutation or deletion of the gene zwf1.

55. The photochemical biosynthesis system of claim 44, wherein the surface membrane is functionalized with a positive charge with a cationic polymer.

56. The photochemical biosynthesis system of claim 55, wherein the cationic polymer is adsorbed onto the surface membrane.

57. The photochemical biosynthesis system of claim 56, wherein the cationic polymer is poly(allylamine) hydrochloride (PAH).

58. The photochemical biosynthesis system of claim 44, wherein the photocatalytic nanoparticles are semiconductor nanoparticles, polymeric nanoparticles, or magnetic nanoparticles.

59. The photochemical biosynthesis system of claim 58, wherein the semiconductor nanoparticles comprise indium phosphide (InP) nanoparticles.

60. The photochemical biosynthesis system of claim 58, wherein the semiconductor nanoparticles have a direct band gap of no higher than 2.0 eV.

61. The photochemical biosynthesis system of claim 44, wherein the photocatalytic nanoparticles are functionalized with a polyphenol.

62. The photochemical biosynthesis system of claim 44, wherein the plurality of functionalized photocatalytic nanoparticles forms a combination of hydrogen and hydrophobic interactions with the chemically modified surface membrane.

63. The photochemical biosynthesis system of claim 44, wherein the plurality of functionalized photocatalytic nanoparticles forms interparticle interactions with one another via metal ion ligand coordination.

64. The photochemical biosynthesis system of claim 63, wherein the metal ion ligand is selected from Ce³⁺, Al³⁺, Fe³⁺, Zn²⁺, Zr⁴⁺, and combinations thereof.

65. The photochemical biosynthesis system of claim 44, wherein the optical density ratio of the plurality of functionalized photocatalytic nanoparticles over the biological cell at 595-600 nm is about 1.2-5.0.

66. A biological hybrid photochemical biosynthesis system, comprising:
a yeast cell having a chemically modified surface membrane, wherein the yeast cell comprises:
an engineered metabolic pathway that utilizes the chemical energy to produce one or more metabolites;
a plurality of functionalized photocatalytic nanoparticles assembled on the chemically modified surface membrane to enable the yeast cell to absorb and convert light energy into chemical energy.

67. The photochemical biosynthesis system of claim 66, wherein the chemical energy is generated in the form of NADPH.

68. The photochemical biosynthesis system of claim 66, wherein the yeast cell is genetically modified to enhance carbon flux for the production of one or more metabolites in the engineered metabolic pathway.

69. The photochemical biosynthesis system of claim 68, wherein the carbon flux is enhanced via overexpression of at least one gene selected from TKL1, RKI1, ADH1, PGK1, aro4, aro4_{K229L}, aro1, and aro1_{D290A}.

70. The photochemical biosynthesis system of claim 66, wherein the engineered metabolic pathway is selected from shikimic acid pathway, flavonoid pathway, stilbenoid pathway, benzylisoquinoline alkaloid pathway, and combinations thereof.

71. The photochemical biosynthesis system of claim 68, wherein the yeast cell is further genetically modified to at least partially disrupt the pentose phosphate pathway.

72. The photochemical biosynthesis system of claim 66, wherein the surface membrane is functionalized with a positive charge with a cationic polymer.

73. The photochemical biosynthesis system of 72, wherein the cationic polymer is adsorbed onto the surface membrane.

74. The photochemical biosynthesis system of claim 72, wherein the cationic polymer is poly(allylamine) hydrochloride (PAH).

75. The photochemical biosynthesis system of claim 66, wherein the photocatalytic nanoparticles are semiconductor nanoparticles.

76. The photochemical biosynthesis system of claim 75, wherein the semiconductor nanoparticles comprise indium phosphide (InP) nanoparticles.

77. The photochemical biosynthesis system of claim 72, wherein the semiconductor nanoparticles have a direct band gap of no higher than 2.0 eV.

78. The photochemical biosynthesis system of claim 66, wherein the photocatalytic nanoparticles are functionalized with a polyphenol.

79. The photochemical biosynthesis system of claim 66, wherein the plurality of functionalized photocatalytic nanoparticles forms a combination of hydrogen and hydrophobic interactions with the chemically modified surface membrane.

80. The photochemical biosynthesis system of claim 66, wherein the plurality of functionalized photocatalytic nanoparticles forms interparticle interactions with one another via metal ion ligand coordination.

81. The photochemical biosynthesis system of claim 80, wherein the metal ion ligand is selected from Ce³⁺, Al³⁺, Fe³⁺, Zn²⁺, Zr⁴⁺, and combinations thereof.

82. The photochemical biosynthesis system of claim 66, wherein the optical density ratio of the plurality of functionalized photocatalytic nanoparticles over the biological cell at 595-600 nm is about 1.2-5.0.

83. A biological hybrid cell system, comprising:
a biological cell having a surface membrane that is chemically modified with a cationic polymer; and
a plurality of polyphenol-functionalized nanoparticles assembled on the chemically modified surface membrane.

84. The cell system of claim 83, wherein the cationic polymer is selected from poly(allylamine) hydrochloride (PAH), poly(ethyleneimine) (PEI), poly-L-(lysine) (PLL), poly[2-(N,N-dimethylamino)ethyl methacrylate] (PDMAEMA), polyethylene glycol-PLL (PEG-PLL), PLL-g-dextran, polyamido amine (PAA), poly(amino-co-ester), poly(N-isopropylacrylamide (PNIPAM), cationic chitosan, cationic dextran, cationic cyclodextrin, cationic gelatin, cationic cellulose, and copolymers thereof.

85. The cell system of claim 84, wherein the cationic polymer is poly(allylamine) hydrochloride (PAH).

86. The cell system of claim 83, wherein the polyphenol-functionalized nanoparticles are selected from the group consisting of polymer nanoparticles, semiconductor nanoparticles, metallic nanoparticles, electromagnetic nanoparticles, magnetic nanoparticles, fluorescent nanoparticles, radioactive nanoparticles, energy conversion nanoparticles, nanoparticles suitable for use in electronics, and a combination thereof.

87. The cell system of claim 83, wherein the plurality of functionalized nanoparticles forms a combination of hydrogen and hydrophobic interactions with the chemically modified surface membrane.

88. The cell system of claim 83, wherein the plurality of functionalized nanoparticles forms interparticle interactions with one another via metal ion ligand coordination.

89. The cell system of claim 88, wherein the metal ion ligand is selected from Ce³⁺, Al³⁺, Fe³⁺, Zn²⁺, Zr⁴⁺, and combinations thereof.

90. The cell system of claim 83, wherein the optical density ratio of the plurality of functionalized photocatalytic nanoparticles over the biological cell at 595-600 nm is about 1.2-5.0.

91. A method of producing a metabolite, comprising exposing the photochemical biosynthesis system of any one of claims 44-82 to a light source.

92. The method of claim 91, wherein the light source is sunlight.

93. The method of claim 91, wherein the metabolite is selected from shikimic acid, a flavonoid, a stilbenoid, a benzylisoquinoline alkaloid, and combinations thereof.

94. A method of converting light energy into chemical energy, comprising exposing the cell system of any one of claims 1-43 or the photochemical biosynthesis system of any one of claims 44-82 to a light source.

95. The method of claim 94, wherein the light source is sunlight.

96. A method of preparing the cell system of any one of claims 1-43 or the photochemical biosynthesis system of any one of claim 44-82, comprising:
preparing the functionalized photocatalytic nanoparticles by adding a functionalization agent to a solution comprising photocatalytic nanoparticles;
chemically modifying the surface membrane of the cell; and
mixing the functionalized photocatalytic nanoparticles and the cell.

97. The method of claim 96, further comprising adding a metal ion to the solution comprising photocatalytic nanoparticles.

98. The method of claim 97, wherein the metal ion is selected from Ce³⁺, Al³⁺, Fe³⁺, Zn²⁺, Zr⁴⁺, and combinations thereof.

99. The method of claim 96, further comprising increasing the pH of the solution comprising photocatalytic nanoparticles after addition of the functionalization agent.

100. The method of claim 96, wherein the functionalization agent is a polyphenol.

101. The method of claim 96, wherein the surface membrane of the cell is chemically modified with a cationic polymer.

102. The method of claim 101, wherein the cationic polymer is adsorbed onto the surface membrane.

103. A method of modifying a biological cell, comprising:
chemically modifying the surface membrane of the cell with a cationic polymer; and
mixing the cell with polyphenol-functionalized nanoparticles;
wherein the functionalized nanoparticles assemble on the chemically modified surface membrane.

104. The method of claim 103, further comprising preparing the polyphenol-functionalized nanoparticles by adding the polyphenol to a solution comprising nanoparticles.

105. The method of claim 104, further comprising adding a metal ion to the solution comprising nanoparticles.

106. The method of claim 104, wherein the metal ion is selected from Ce³⁺, Al³⁺, Fe³⁺, Zn²⁺, Zr⁴⁺, and combinations thereof.

107. The method of claim 104, further comprising increasing the pH of the solution comprising photocatalytic nanoparticles after addition of the polyphenol.

108. The method of claim 103, wherein the cationic polymer is poly(allylamine) hydrochloride (PAH).

109. The method of claim 103, wherein the cationic polymer is adsorbed onto the surface membrane.
